SEQUENCE LISTING (4 sequences)

SEQ ID NO: 1 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0325666 A1
(43) Pub. Date: Oct. 23, 2025

(54) PHARMACEUTICAL COMPOSITIONS CONTAINING ANTI-HER2 ANTIBODY FOR SUBCUTANEOUS ADMINISTRATION

(71) Applicant: EIRGENIX, INC., New Taipei City (TW)

(72) Inventors: Ching-Yi CHOU, New Taipei City (TW); Tsan-Hui WU, New Taipei City (TW)

(21) Appl. No.: 18/857,196

(22) PCT Filed: Apr. 21, 2023
(86) PCT No.: PCT/CN2023/089632

§ 371 Date: Oct. 16, 2024

Related U.S. Application Data
(60) Provisional application No. 63/333,930, filed on Apr. 22, 2022.
(60) Provisional application No. 63/333,935, filed on Apr. 22, 2022.

(51) Int. Cl. A61K 39/395; A61K 9/00; A61K 39/00; A61K 47/18; A61K 47/20; A61K 47/26; A61P 35/00; C07K 16/32

(52) U.S. Cl. CPC A61K 39/39591; A61K 9/0019; A61K 47/183; A61K 47/20; A61K 47/26; A61P 35/00; C07K 16/32; A61K 2039/505; C07K 2317/24

(57) ABSTRACT

The present invention relates to stable liquid pharmaceutical formulations containing a high concentration of trastuzumab, pertuzumab or a mixture thereof for convenient subcutaneous administration. Formulations of the present invention can be administered to treat cancer, such as breast cancer and metastatic gastric or gastroesophageal junction adenocarcinoma.

### CROSS REFERENCE TO RELATED APPLICATION

This application claims the benefit of U.S. Provisional Application No. 63/333,930 and U.S. Provisional Application No. 63/333,935 filed Apr. 22, 2022, the entireties of which are incorporated by reference.

### INCORPORATION BY REFERENCE OF MATERIAL IN ASCII TEXT FILE

This application incorporates by reference the Sequence Listing contained in the following file: File name: P4330-PCT_seq listing; 6 KB, created Apr. 14, 2023.

### BACKGROUND

The HER2 (human epidermal growth factor receptor 2) proto-oncogene encodes a transmembrane protein. HER2 protein is overexpressed by many adenocarcinomas, such as breast cancer (for example: breast adenocarcinomas), gastric cancer (for example: stomach adenocarcinoma), and gastroesophageal cancer (for example: gastroesophageal junction adenocarcinoma). The overexpression of HER2 protein may be exploited by targeting it with a monoclonal antibody (MAb) specific for HER2, thereby making it possible to treat diseases such as HER2-positive breast cancer and gastrointestinal cancer. Trastuzumab is a recombinant humanized monoclonal antibody directed against HER2. After binding to HER2 on the tumor cell surface, trastuzumab induces an antibody-dependent cell-mediated cytotoxicity against tumor cells that overexpress HER2 protein.

The pharmaceutical use of monoclonal antibodies has increased over the past years. In many instances such monoclonal antibodies have been injected via the intravenous (IV) route. Most therapeutic monoclonal antibodies (mAbs) have been delivered via conventional intravenous (IV) administration. IV administration requires patients to travel long distances to a medical facility with medical professionals trained to administer IV infusions and determine appropriate doses and infusion rates. Clinical administration is both expensive and inconvenient for patients.

Furthermore, the amount of time needed to inject a suitable amount of typically low-concentration monoclonal antibodies through IV infusions is long (approximately 90 mins), and is inconvenient to patients (requiring visiting well-staffed clinics).

Alternative administration pathways include subcutaneous (SC) administration which allows therapeutics to be conveniently and economically self-administered by patients at home. This is more convenient for patients, which can lead to higher compliance, and less expense for healthcare providers.

However, the amount of monoclonal antibodies that can be injected via the subcutaneous route is limited (less than 1 to 2 mL), and a liquid formulation having suitable solubility and stability is accordingly needed. Thus, there has been a need to address SC administration for targeted therapies with stable liquid pharmaceutical formulations containing high concentrations of antibodies such as trastuzumab.

### SUMMARY

An embodiment of the invention is a pharmaceutical composition including anti human epidermal growth factor receptor-2 (HER2) protein, a buffering agent at pH 4.6 to 6.5, one or more stabilizers and a tonicity agent.

In an embodiment of the invention is a pharmaceutical composition including an anti-HER2 antibodies, sucrose, and methionine.

In yet another embodiment of the invention is a pharmaceutical composition including an active pharmaceutical ingredient including anti-HER2 antibodies, methionine, and glutamic acid.

According to an embodiment of the invention, the anti-HER2 antibodies includes trastuzumab or pertuzumab.

According to an embodiment of the invention, the anti-HER2 antibodies are trastuzumab or pertuzumab, and the trastuzumab or pertuzumab of the pharmaceutical composition has a concentration from 80 to 150 mg/mL.

According to an embodiment of the invention, the stabilizers including methionine, glutamic acid, arginine, N-alpha-acetylarginine, proline, glycine, lysine, glutamine, or a combination thereof.

According to an embodiment of the invention, the methionine of the pharmaceutical composition has a concentration from 5 to 15 mM and the glutamic acid has a concentration from 5 to 15 mM.

According to an embodiment of the invention, the pharmaceutical composition further includes a tonicity agent selected from trehalose, sucrose, mannitol, or sorbitol.

According to an embodiment of the invention, the tonicity agent of the pharmaceutical composition is sucrose.

According to an embodiment of the invention, the sucrose has a concentration from 150 to 300 mM.

According to an embodiment of the invention, the pharmaceutical composition is substantially free of hyaluronidase.

According to an embodiment of the invention, the pharmaceutical composition is configured for subcutaneous administration.

According to an embodiment of the invention, the pharmaceutical composition has a FcγRIII binding of 93.9 to 107.9 relative potency (%) after storage for 3 days at a temperature of 5° C.±3° C.

According to an embodiment of the invention, the pharmaceutical composition includes: (a) from 5 to 150 mg/mL trastuzumab or pertuzumab; (b) from 0 to 15 mM methionine; (c) from 0 to 15 mM glutamic acid; (d) from 0 to 0.05% (w/v) polysorbate 80; (e) from 0 to 300 mM sucrose; and (f) from 5 to 40 mM acetate buffer at pH 4.6 to 6.5.

According to an embodiment of the invention, the pharmaceutical composition includes: (a) from 80 to 150 mg/mL trastuzumab or pertuzumab; (b) from 5 to 15 mM methionine; (c) from 5 to 15 mM glutamic acid; (d) from 0.01 to 0.03% (w/v) polysorbate 80; (e) from 200 to 220 mM sucrose; and (f) from 5 to 40 mM acetate buffer at pH 4.6 to 6.5.

Another embodiment of the invention is a method of treating cancer in a subject in need thereof comprising administering subcutaneously to the subject a therapeutically effective amount of the pharmaceutical composition.

According to an embodiment of the invention, the cancer is breast cancer.

According to an embodiment of the invention, the cancer is metastatic gastric or gastroesophageal junction adenocarcinoma.

According to an embodiment of the invention, the pharmaceutical composition is administered once every three weeks.

Another embodiment of the invention is a prefilled syringe including 3 mL to 7 mL of a solution. The solution includes: (a) trastuzumab, pertuzumab or their mixture; (b) methionine; and (c) glutamic acid.

Another embodiment of the invention is a prefilled syringe including 3 mL to 7 mL of a solution. The solution includes: (a) trastuzumab or pertuzumab; (b) methionine; (c) glutamic acid; (d) organic co-solvents; (e) sucrose; and (f) acetate buffer.

According to an embodiment of the invention, the trastuzumab or pertuzumab has a concentration from 80 to 150 mg/mL.

According to an embodiment of the invention, the methionine has a concentration from 5 to 15 mM and the glutamic acid has a concentration from 5 to 15 mM.

According to an embodiment of the invention, the solution further includes a sucrose.

Another embodiment of the invention is a method of treating cancer in a subject in need thereof including administering subcutaneously to the subject a therapeutically effective amount of the solution from a prefilled syringe.

Another embodiment of the invention is a method of stabilizing trastuzumab including combining the trastuzumab with methionine and glutamic acid in a solution. The composition has a FcγRIII binding affinity of 93.9 to 107.9 relative potency (%) after storage for 3 days at a temperature of 5° C.±3° C.

Within this specification embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention described herein.

### DESCRIPTION OF THE DRAWINGS

FIG. 1 displays amino acid sequences for the light chain of trastuzumab (SEQ ID NO: 1) and the heavy chain of trastuzumab (SEQ ID NO: 2) with their corresponding domains.

FIG. 2 Colloidal stability of high-concentrated trastuzumab in different buffers. Scattering constant vs. trastuzumab concentration. FIG. 2A includes Histidine buffer at pH 6.5. FIG. 2B includes Histidine buffer at pH 6.0. FIG. 2C includes Citric acid buffer at pH 6.0. FIG. 2D includes Citric acid buffer at pH 5.5. FIG. 2E includes Acetic acid buffer at pH 5.5. FIG. 2F includes Acetic acid buffer at pH 5.0.

FIG. 3 Stability of high-concentrated trastuzumab in an acetic buffer as measured by Size Exclusion Chromatography. FIG. 3 shows the antibody profile of high-concentrated trastuzumab over 28 days under accelerated thermal stress of 40° C. FIG. 3A is the antibody profile under Acetic acid at pH 5.4. FIG. 3B is antibody profile under Acetic acid at pH 5.0. FIG. 3C is the antibody profile under Acetic acid at pH 4.6.

FIG. 4 Stability of high-concentrated trastuzumab in acetic buffer as measured by Cation Exchange Chromatography. FIG. 4 shows the antibody charge variants profile of high-concentrated trastuzumab over 28 days under accelerated thermal stress of 40° C. FIG. 4A shows the charge variant profile under Acetic acid at pH 5.4. FIG. 4B shows the charge variant profile under Acetic acid at pH 5.0. FIG. 4C shows the charge variant profile under Acetic acid at pH 4.6.

FIG. 5 Long-term stability of trastuzumab formulation as measured by SEC-HPLC analysis. FIG. 5 shows the antibody integrity of trastuzumab up to 24 months under different temperatures. FIG. 5A shows the antibody purity under 5° C. FIG. 5B shows the antibody purity under 25° C. FIG. 5C shows the antibody purity under 40° C.

FIG. 6 Long-term stability of trastuzumab formulation as measured by CEX analysis. FIG. 6 shows the antibody main peak profile of trastuzumab up to 24 months under different temperatures. FIG. 6A shows the main peak percentage under 5° C. FIG. 6B shows the main peak percentage under 25° C. FIG. 6C shows the main peak percentage under 40° C.

FIG. 7 Stability of trastuzumab formulation as measured by SEC-HPLC analysis. FIG. 7 shows the antibody integrity of trastuzumab up to 3 months under different temperatures. FIG. 7A shows the antibody purity under 5° C. FIG. 7B shows the antibody purity under 25° C. FIG. 7C shows the antibody purity under 40° C.

FIG. 8 Stability of trastuzumab formulation as measured by CEX analysis. FIG. 8 shows the antibody main peak profile of trastuzumab up to 3 months under different temperatures. FIG. 8A shows the main peak percentage under 5° C. FIG. 8B shows the main peak percentage under 25° C. FIG. 8C shows the main peak percentage under 40° C.

FIG. 9 Stability of pertuzumab formulation as measured by SEC-HPLC analysis. FIG. 9 shows the antibody integrity of pertuzumab up to 3 months under different temperatures. FIG. 9A shows the antibody purity under 5° C. FIG. 9B shows the antibody purity under 25° C. FIG. 9C shows the antibody purity under 40° C.

FIG. 10 Stability of pertuzumab formulation as measured by CEX analysis. FIG. 10 shows the antibody charge heterogeneity profile of pertuzumab up to 3 months under different temperatures. FIG. 10A shows the charge heterogeneity profile of sample Example 41. FIG. 10B shows the charge heterogeneity profile of sample Example 42. FIG. 10C shows the charge heterogeneity profile of sample Example 43. FIG. 10D shows the charge heterogeneity profile of sample Example 44.

FIG. 11. Antitumor activity of trastuzumab in different buffer conditions. Mean values of tumor volume+/−SE (mm³) plotted on the y-axis; number of days post tumor implant plotted on the x-axis. PBS buffer control—filled square, IV trastuzumab-filled circles, and high-concentrated trastuzumab-3 -filled triangles.

FIG. 12. Pharmacokinetic (PK) study to test plasma concentrations of trastuzumab (g/mL) vs. time in CD-1 mice following subcutaneous or intravenous administration of trastuzumab in various formulations at 10 mg/kg. The compounds high-concentrated trastuzumab and IV trastuzumab contain the same active pharmaceutical ingredient (API) (Trastuzumab). Concentration was based on median values. G3: Group 3, high-concentrated trastuzumab SC. G5: Group 5, IV trastuzumab IV. FIG. 12A. Timepoints of observations were from 0 to 96 hours. FIG. 12B. Timepoints of observations were from 0.25 to 1344 hours.

### DETAILED DESCRIPTION

The present invention relates to stable liquid pharmaceutical formulations containing a high-concentration of anti-HER2 antibodies including trastuzumab, pertuzumab or a mixture thereof for the convenient subcutaneous administration of a low volume of the formulation.

### Anti-HER2 Antibody

Human epidermal growth factor receptor 2 (HER2) is a tyrosine-phosphorylating enzyme that binds to the surface of cell membranes. HER2 protein is known to be involved in a signaling pathway that causes cell growth and cell differentiation, as well as in cell malignancy upon overexpression or activation. The overexpression of HER2 protein may be inhibited using a targeted therapeutic agent, which includes anti-HER2 antibodies. The anti-HER2 antibodies include trastuzumab and pertuzumab, but are not limited thereto. An application of inhibiting overexpression of HER2 also involves using a mixture of trastuzumab and pertuzumab, but not limited thereto.

### Trastuzumab

Trastuzumab is a recombinant humanized monoclonal antibody directed against HER2. The amino acid sequence for the light chain of trastuzumab is: (SEQ ID NO: 1).

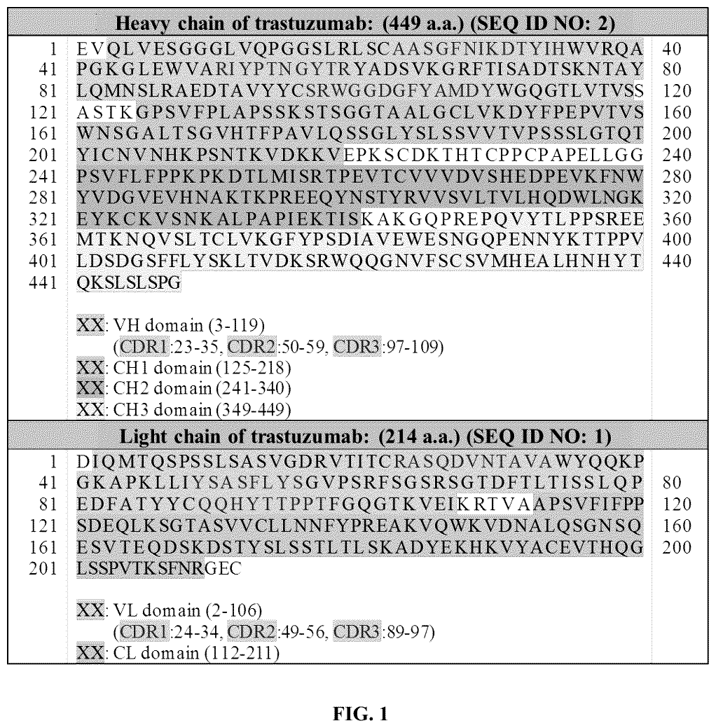

The amino acid sequence for the heavy chain of trastuzumab is: (SEQ ID NO: 2). FIG. 1 displays amino acid sequences for the light chain of trastuzumab (SEQ ID NO: 1) and the heavy chain of trastuzumab (SEQ ID NO:2) with their corresponding domains.

Trastuzumab is CAS Number 180288-69-1.

### Pertuzumab

Pertuzumab is a recombinant humanized monoclonal antibody directed against HER2. The amino acid sequence for the light chain of pertuzumab is: (SEQ ID NO: 3). The amino acid sequence for the heavy chain of pertuzumab is: (SEQ ID NO: 4). Pertuzumab is CAS Number 380610-27-5.

The concentration of trastuzumab or pertuzumab may be 5 to 150 mg/mL, 10 to 150 mg/mL, 20 to 150 mg/mL, 30 to 150 mg/mL, 40 to 150 mg/mL, 50 to 150 mg/mL, 60 to 150 mg/mL, 70 to 150 mg/mL, 80 to 150 mg/mL, 90 to 150 mg/mL, 100 to 150 mg/mL, 110 to 150 mg/mL, 120 to 150 mg/mL, 130 to 150 mg/mL, or 140 to 150 mg/mL. Moreover, the concentration of trastuzumab may be 95 to 145 mg/mL, 100 to 140 mg/mL, 105 to 135 mg/mL, 110 to 130 mg/mL, 115 to 125 mg/mL, or 120 mg/mL.

The concentration of the trastuzumab or pertuzumab may be freely adjusted within a range that does not substantially adversely affect the stability of the stable liquid pharmaceutical formulation according to the present invention.

As used herein, “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, glutamic acid or malonic acid or by using other methods used in the art such as ion exchange. Other pharmaceutically acceptable salts include acetate, glutamine, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

### Pharmaceutical Compositions

A compound provided in accordance with the present invention is usually administered in the form of a liquid pharmaceutical composition. This invention therefore provides pharmaceutical compositions that contain, as the active ingredient, one or more of the compounds described herein, or a pharmaceutically acceptable salt or ester thereof and one or more pharmaceutically acceptable carriers. As used herein, “pharmaceutically acceptable carrier” refers to a non-toxic carrier, adjuvant, excipient, or the like that does not destroy the pharmacological activity of the compound with which it is formulated. Pharmaceutically acceptable carriers may be used in the compositions described herein include, but not limited to, buffers, tonicity agents, stabilizers, organic co-solvents, permeation enhancers, solubilizers, fillers, diluents, and a combination thereof.

A pharmaceutical composition of this invention may be substantially free of hyaluronidase, a penetration enhancer. Hyaluronidase includes all of a variety of commonly known hyaluronidases or modified hyaluronidases, such as the enzyme protein having the amino acid sequence of GenBank: AAC70915.1 in humans. The hyaluronidase enzyme may be a glycoprotein, for example, rHuPH20. The term “substantially free of hyaluronidase” means that there is no detectable hyaluronidase, less than 10 units/dose hyaluronidase, less than 5 units/dose hyaluronidase, or less 1 units/dose in the composition when measured with a technique well-known in the art such as ELISA.

### Pharmaceutical Compositions: Buffers

A pharmaceutical composition may include one or more buffers or buffering agents. The buffer may be L-histidine/histidine-HCl, sodium citrate/citric acid, L-histidine/acetic acid, phosphate, sodium acetate/acetic acid, acetate, or a combination thereof from 2 to 120 mM, 5 to 100 mM, from 5 to 90 mM, from 5 to 80 mM, from 5 to 70 mM, from 5 to 60 mM, from 5 to 50 mM, from 5 to 40 mM, from 5 to 30 mM, from 5 to 20 mM, from 5 to 10 mM, from 5 to 35 mM, from 10 to 30 mM, from 15 to 25 mM, or 20 mM.

The concentration of one of the buffers may be from 2 to 120 mM, 5 to 100 mM, from 5 to 90 mM, from 5 to 80 mM, from 5 to 70 mM, from 5 to 60 mM, from 5 to 50 mM, from 5 to 40 mM, from 5 to 30 mM, from 5 to 20 mM, from 5 to 10 mM, from 5 to 35 mM, from 10 to 30 mM, from 15 to 25 mM, or 20 mM.

The buffer can stabilize the pH of the pharmaceutical composition at a pH of from 4.6 to 6.5, from 4.7 to 6.3, from 4.8 to 6.1, from 4.9 to 5.9, from 5.0 to 5.7, from 5.0 to 5.4, from 5.1 to 5.5, from 5.1 to 5.3, from 4.6 to 5.4, from 4.6 to 5.0, from 5.0 to 5.4, from 5.2 to 5.6, or 5.2.

### Pharmaceutical Compositions: Tonicity Agents

A pharmaceutical composition may include one or more tonicity agents. The tonicity agent may be polyols or sugar derivatives, but not limited thereto. The tonicity agent may be sucrose, trehalose, mannitol, sorbitol, or a combination thereof.

The concentration of one of the tonicity agents may be from 20 to 300 mM, from 50 to 290 mM, from 80 to 280 mM, from 110 to 270 mM, from 140 to 260 mM, from 170 to 250 mM, from 180 to 240 mM, from 190 to 230 mM, from 200 to 220 mM, or 210 mM (7.18% w/v).

### Pharmaceutical Compositions: Stabilizers

A pharmaceutical composition may include one or more stabilizers. The stabilizer may be methionine (Met), glutamic acid (Glu), arginine (Arg), N-alpha-acetylarginine, Proline (Pro), Glycine (Gly), Lysine (Lys), Glutamine (Gln), or a combination thereof such as Met and Glu.

The concentration of one of the stabilizers may be less than or equal to 50 mM, from 5 to 45 mM, from 10 to 40 mM, from 15 to 35 mM, from 20 to 30 mM, from 5 to 40 mM, from 5 to 30 mM, from 5 to 20 mM, from 0 to 20 mM, or 10 mM.

The concentration of Met may be less than or equal to 20 mM, from 3 to 17 mM, from 5 to 15 mM, from 7 to 13 mM, from 0 to 10 mM, or 10 mM.

The concentration of Arg may be from less than or equal to 50 mM, from 0 to 50 mM, from 5 to 40 mM, from 10 to 30 mM, or from 15 to 25 mM.

The concentration of N-alpha-acetylarginine may be from less than or equal to 10 mM, from 0 to 10 mM. or from 3 to 7 mM.

The concentration of Pro may be from less than or equal to 20 mM, from 0 to 20 mM, from 3 to 17 mM, from 5 to 15 mM, from 7 to 13 mM, or 10 mM.

The concentration of Gly may be from less than or equal to 40 mM, from 0 to 40 mM, from 5 to 35 mM, from 5 to 30 mM, from 5 to 25 mM, or from 5 to 20 mM.

The concentration of Lys may be from less than or equal to 20 mM, from 0 to 20 mM, from 3 to 17 mM, from 5 to 15 mM, from 7 to 13 mM, or 10 mM.

The concentration of Gln may be from less than or equal to 20 mM, from 0 to 20 mM, from 3 to 17 mM, from 5 to 15 mM, from 7 to 13 mM, or 10 mM.

The concentration of Glu may be from less than or equal to 20 mM, from 0 to 20 mM, from 3 to 17 mM, from 5 to 15 mM, from 7 to 13 mM, or 10 mM.

### Pharmaceutical Compositions: Organic Co-Solvents

A pharmaceutical composition may include one or more organic co-solvents which may be organic co-solvent polysorbate 80 (PS80), polysorbate 20 (PS20), poloxamer 188, polyethylene glycol (PEG), propylene glycol or a combination thereof. In some embodiments, organic co-solvents may also be referred as surfactants.

The concentration of one of the organic co-solvents may be less than or equal to 0.05% (w/v), from 0 to 0.05% (w/v), from 0.01 to 0.04% (w/v), from 0.01 to 0.03% (w/v), or 0.02% (w/v).

### Pharmaceutical Compositions: Other Components

A pharmaceutical composition may contain histidine, citrate, phosphate, or a mixture thereof. In an embodiment, the pharmaceutical composition may include histidine in the buffer of the liquid formulation.

A pharmaceutical composition may include salts. The salt may be, but not limited to, NaCl, KCl, KBr, NaBr, Na₂SO₄, NaSCN, K₂SO₄, and the like, or a mixture thereof. The concentration of salt may be less than or equal to 155 mM, less than or equal to 150 mM, from 10 to 145 mM, from 20 to 135 mM, from 30 to 125 mM, from 40 to 115 mM, from 50 to 100 mM, from 60 to 90 mM, or from 70 to 80 mM. Similarly, the salt concentration may be from 0.7% (w/v) to 1.1% (w/v).

A pharmaceutical composition may include a preservative. The preservative may include, but not limited to, octadecyl dimethylbenzyl ammonium chloride, benzalkonium chloride, benzethonium chloride, phenol, butyl alcohol, benzyl alcohol, alkyl paraben, catechol, resorcinol, cyclohexanol, 3-pentanol, m-cresol, and the like, or a mixture thereof.

The pharmaceutical composition may also include an additive known in the art within a concentration range that does not substantially adversely affect the activity of the antibody or the useability of the formulation.

### Pharmaceutical Compositions: Component Combinations

Below is a list of contemplated component combinations for formulations containing anti-HER2 antibodies including trastuzumab, pertuzumab or a mixture thereof. For example, the formulation may contain trastuzumab or pertuzumab at 120 mg/mL.

A formulation including the combination of acetic acid buffer and sucrose;

A formulation including acetic acid, sucrose and PS80;

A formulation including acetic acid, sucrose, and the combination of methionine and glutamic acid;

A formulation including acetic acid, sucrose, PS80 and the combination of methionine and glutamic acid;

A formulation including acetic acid buffer, sucrose, PS80, and methionine;

A formulation including acetic acid buffer, sucrose, PS80, and glutamic acid;

A formulation including acetic acid buffer, histidine, sucrose, methionine, and glutamic acid;

A formulation including acetic acid buffer, histidine, sucrose, methionine, and glycine;

A formulation including histidine buffer, trehalose, PS20 and methionine;

A formulation including 20 mM acetic acid buffer at pH 5.2, 210 mM sucrose, 10 mM methionine, 10 mM glutamic acid, and 0.02% PS80.

### Pharmaceutical Compositions: Traits

The following traits describe the pharmaceutical formulations of this present invention. Examples of the techniques used to measure these traits can be found in the Examples section below.

The following list describes a pharmaceutical liquid formulation with particular HER2-binding affinity

A pharmaceutical liquid formulation having a HER2-binding relative potency (%) of 93.9 to 107.9 as measured using an FcγRIII binding affinity assay after storage for 3 days at a temperature of 5±3° C.

A pharmaceutical liquid formulation having a HER2-binding relative potency (%) of 90.0 to 118.0 as measured using an FcγRIII binding affinity assay after storage for 3 days at a temperature of 40±2° C. under accelerated thermal stress condition.

A pharmaceutical liquid formulation having a HER2-binding relative potency (%) of 93.9 to 107.9 as measured using an FcγRIII binding affinity assay after storage for 3 days at a temperature of 5±3° C., and in 20 mM L-Histidine buffer at pH 6.0 or 6.5, 20 mM Citric Acid buffer at pH 5.5 or 6.0, or 20 mM Acetic Acid buffer at pH 5.0 or 5.5.

A pharmaceutical liquid formulation having a HER2-binding relative potency (%) of 90.0 to 118.0 as measured using an FcγRIII binding affinity assay after storage for 3 days at a temperature of 40±2° C. under accelerated thermal stress condition, and in 20 mM L-Histidine buffer at pH 6.0 or 6.5, 20 mM Citric Acid buffer at pH 5.5 or 6.0, or 20 mM Acetic Acid buffer at pH 5.0 or 5.5.

The following list describes a pharmaceutical liquid formulation with a particular amount of main component content (Main peak/Monomer %)

A pharmaceutical liquid formulation including 95 to 99% of a main component, as measured by SEC-HPLC after storage for 28 days at a temperature of 5±3° C.

A pharmaceutical liquid formulation including 95 to 99% of a main component, as measured by SEC-HPLC after storage for 28 days at a temperature of 5±3° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

A pharmaceutical liquid formulation including 94 to 99% of a main component, as measured by SEC-HPLC after storage for 28 days at a temperature of 40±2° C.

A pharmaceutical liquid formulation including 94 to 99% of a main component, as measured by SEC-HPLC after storage for 28 days at a temperature of 40±2° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

The following list describes a pharmaceutical liquid formulation with a particular amount of high-molecular-weight component (a peak in which the retention time thereof is located before a main peak)

A pharmaceutical liquid formulation including 0.78 to 2.17% of a high-molecular-weight component, as measured by SEC-HPLC after storage for 28 days at a temperature of 5±3° C.

A pharmaceutical liquid formulation including 0.78 to 2.17% of a high-molecular-weight component, as measured by SEC-HPLC after storage for 28 days at a temperature of 5±3° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

A pharmaceutical liquid formulation including 0.78 to 4.11% of a high-molecular-weight component, as measured by SEC-HPLC after storage for 28 days at a temperature of 40±2° C.

A pharmaceutical liquid formulation including 94 to 99% of a main component, as measured by SEC-HPLC after storage for 28 days at a temperature of 40±2° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

The following list describes a pharmaceutical liquid formulation with a particular amount of low-molecular-weight component (a peak in which the retention time thereof is located after a main peak)

A pharmaceutical liquid formulation including 0% of a low-molecular-weight component, as measured by SEC-HPLC after storage for 28 days at a temperature of 5±3° C.

A pharmaceutical liquid formulation including 0% of a low-molecular-weight component, as measured by SEC-HPLC after storage for 28 days at a temperature of 5±3° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

A pharmaceutical liquid formulation including 1.61 to 2.20% of a low-molecular-weight component, as measured by SEC-HPLC after storage for 28 days at a temperature of 40±2° C.

A pharmaceutical liquid formulation including 1.61 to 2.20% of a low-molecular-weight component, as measured by SEC-HPLC after storage for 28 days at a temperature of 40±2° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

The following list describes a pharmaceutical liquid formulation with a particular amount of a main species of the charged variant components

A pharmaceutical liquid formulation including 55.06 to 64.58% of a main species charged variant component, as measured by CIX-HPLC after storage for 28 days at a temperature of 5±3° C.

A pharmaceutical liquid formulation including 55.06 to 64.58% of a main species charged variant component, as measured by CIX-HPLC after storage for 28 days at a temperature of 5±3° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

A pharmaceutical liquid formulation including 12.71 to 64.15% of a main species charged variant component, as measured by CIX-HPLC after storage for 28 days at a temperature of 40±2° C.

A pharmaceutical liquid formulation including 12.71 to 64.15% of a main species charged variant component, as measured by CIX-HPLC after storage for 28 days at a temperature of 40±2° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

The following list describes a pharmaceutical liquid formulation with a particular amount of an acidic species of the charged variant components (a peak in which the retention time thereof is located before a main species charged variant)

A pharmaceutical liquid formulation including 25.98 to 59.69% of an acidic species charged variant component, as measured by CIX-HPLC after storage for 28 days at a temperature of 5±3° C.

A pharmaceutical liquid formulation including 25.98 to 59.69% of an acidic species charged variant component, as measured by CIX-HPLC after storage for 28 days at a temperature of 5±3° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

A pharmaceutical liquid formulation including 39.20 to 71.02% of an acidic species charged variant component, as measured by CIX-HPLC after storage for 28 days at a temperature of 40±2° C.

A pharmaceutical liquid formulation including 39.20 to 71.02% of an acidic species charged variant component, as measured by CIX-HPLC after storage for 28 days at a temperature of 40±2° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

The following list describes a pharmaceutical liquid formulation with a particular amount of a basic species of the charged variant components (a peak in which the retention time thereof is located after a main species charged variant)

A pharmaceutical liquid formulation including 8.47 to 12.56% of a basic species charged variant component, as measured by CIX-HPLC after storage for 28 days at a temperature of 5±3° C.

A pharmaceutical liquid formulation including 8.47 to 12.56% of a basic species charged variant component, as measured by CIX-HPLC after storage for 28 days at a temperature of 5±3° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

A pharmaceutical liquid formulation including 16.27 to 34.50% of a basic species charged variant component, as measured by CIX-HPLC after storage for 28 days at a temperature of 40±2° C.

A pharmaceutical liquid formulation including 16.27 to 34.50% of a basic species charged variant component, as measured by CIX-HPLC after storage for 28 days at a temperature of 40±2° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

The following list describes a pharmaceutical liquid formulation with a particular turbidity

A pharmaceutical liquid formulation having an absorbance A350 of 0.23 to 0.338 as measured using a spectrophotometer (for example, SpectraMax® iD3 Multi-Mode Microplate reader) after storage for 28 days at a temperature of 5° C.±3° C.

A pharmaceutical liquid formulation having an absorbance A350 of 0.23 to 0.338 as measured using a spectrophotometer (for example, SpectraMax® iD3 Multi-Mode Microplate reader) after storage for 28 days at a temperature of 5±3° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

A pharmaceutical liquid formulation having an absorbance A350 of 0.232 to 0.397 as measured using a spectrophotometer (for example, SpectraMax® iD3 Multi-Mode Microplate reader) after storage for 28 days at a temperature of 40° C.±2° C.

A pharmaceutical liquid formulation having an absorbance A350 of 0.232 to 0.397 as measured using a spectrophotometer (for example, SpectraMax® iD3 Multi-Mode Microplate reader) after storage for 28 days at a temperature of 40±2° C. in 20 mM L-Histidine buffer at pH 6.0 or 6.5, 20 mM Citric Acid buffer at pH 5.5 or 6.0, or 20 mM Acetic Acid buffer at pH 5.0 or 5.5.

The following list describes a pharmaceutical liquid formulation with a particular colloidal stability of the API in buffers.

Dynamic Light Scattering (DLS) was used to measure the colloidal stability, particle size and aggregation of the API in buffers.

A positive slope is measured by using DLS to determine the API aggregation and colloidal stability in Histidine buffer. Samples are measured in increasing concentration of the API in Histidine buffer at pH 6.0 or pH 6.5.

A negative slope is measured by using DLS to determine the API aggregation and colloidal stability in Citrate buffer. Samples are measured in increasing concentration of the API in Citrate buffer at pH 5.5 or pH 6.0.

A positive slope is measured by using DLS to determine the API aggregation and colloidal stability in Acetate buffer. Samples are measured in increasing concentration of the API in Acetate buffer at pH 5.0 or pH 5.5.

### Pharmaceutical Compositions: Preparation

Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.).

Sterile injectable solutions or intravenous fluid may be prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the methods of preparation can be vacuum-drying and freeze-drying techniques which yield a powder of the active ingredient plus any additional desired ingredient from a previously sterile-filtered solution thereof.

A liquid formulation of highly concentrated trastuzumab can be made from IV trastuzumab that has been reprocessed via ultrafiltration (UF) and diafiltration (DF) using methods well-known in the art. =Ultra-centrifugal filters and tangential flow filtration (TFF) are two approaches to achieve buffer exchange and concentrate mAbs. Buffer exchange can also be carried out via dialysis tubing or a dialysis cassette

### Methods of Treatment and Routes of Administration

The present invention provides a method of treating a subject in need thereof comprising administering a therapeutically effective amount of a pharmaceutical composition described herein to the subject.

The present invention further provides a method of treating cancer in a subject in need thereof comprising administering to the subject a therapeutically effective amount of a pharmaceutical composition described herein.

The present invention further provides a method of binding HER2 in a subject in need thereof comprising administering to the subject a therapeutically effective amount of a pharmaceutical composition described herein.

The methods described herein further comprise identifying a subject having cancer prior to the administration of a pharmaceutical composition described herein.

The present invention also provides for:
(1) A pharmaceutical composition described herein for use in a method for treating a subject by therapy.
(2) A pharmaceutical composition described herein for use in a method of treating cancer, the method comprising administering a therapeutically effective amount of the pharmaceutical composition to a subject in need thereof.
(3) A pharmaceutical composition described herein for use in binding HER2, wherein binding HER2 comprises administering a therapeutically effective amount of the composition to a subject in need thereof.
(4) The use of a therapeutically effective amount of a pharmaceutical composition described herein to treat a subject in need thereof.
(5) The use of a pharmaceutical composition described herein to treat cancer in a subject in need thereof, wherein the treatment comprises administering a therapeutically effective amount of the pharmaceutical composition to the subject.
(6) The use of a pharmaceutical composition described herein for binding HER2, wherein binding HER2 comprises administering a therapeutically effective amount of the composition to a subject in need thereof.
(7) The use of a pharmaceutical composition described herein for the manufacture of a medicament.
(8) The use of a pharmaceutical composition described herein for the manufacture of a medicament for use in treating cancer in a subject in need thereof, wherein the treatment comprises administering a therapeutically effective amount of the composition to the subject.
(9) The use of a pharmaceutical composition described herein for the manufacture of a medicament for use in binding HER2, wherein binding HER2 comprises administering a therapeutically effective amount of the composition to a subject in need thereof.

As used herein, and unless otherwise specified, the terms “treat,” “treating” and “treatment” contemplate an action that occurs while a subject is suffering from the specified disease, disorder, or condition, which reduces the severity of the disease, disorder or condition, or retards or slows the progression of the disease, disorder or condition (“therapeutic treatment”). Disease, disorder, and condition are used interchangeably herein.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

As used herein, and unless otherwise specified, a “therapeutically effective amount” of a compound is an amount sufficient to provide a therapeutic benefit in the treatment of a disease, disorder, or condition, or to delay or minimize one or more symptoms associated with the disease, disorder, or condition. A therapeutically effective amount of a compound means an amount of therapeutic agent which provides a therapeutic benefit in the treatment of the disease, disorder, or condition. The term “therapeutically effective amount” can encompass an amount that improves overall therapy or reduces or avoids symptoms or causes of a disease or condition.

The pharmaceutical compositions may be administered to treat cancer such as breast cancer. Types of breast cancer include adjuvant breast cancer, metastatic breast cancer, advanced breast cancer, and early-stage breast cancer. The pharmaceutical compositions may also be administered to treat cancer such as metastatic gastric or gastroesophageal junction adenocarcinoma as well as ovarian, stomach, colon, and gastrointestinal cancer.

The pharmaceutical compositions may be administered in either single or multiple doses by any of the accepted modes of administration of agents having similar utilities, for example as described in those patents and patent applications incorporated by reference, including parenterally such as subcutaneously.

The amount of trastuzumab to be administered subcutaneously per dose can be from 100 mg to 1000 mg, from 200 mg to 900 mg, from 300 mg to 800 mg, from 300 mg to 750 mg, or from 400 mg to 700 mg, from 480 mg to 700 mg, from 500 to 700 mg, from 550 to 650 mg, from 500 mg to 600 mg, 600 mg, or 750 mg. Each subcutaneous dose can be from 0.5 mL to 10 mL, from 1 mL to 9 mL, from 2 mL to 8 mL, 3 mL to 7 mL, or from 4 mL to 6 mL, or 5 mL in volume.

For subcutaneous administration, patients should be treated once every week, once every two weeks, once every three weeks, once every four weeks, or once every five weeks. The dose should be administered over 2-5 minutes and preferably injected in different sites of the thighs. Patients with early breast cancer should be treated with trastuzumab subcutaneously for 52 weeks or until disease recurrence or unacceptable cardiac toxicity whichever occurs first. Patients with metastatic breast cancer (MBC) should be treated with trastuzumab subcutaneously until the progression of disease.

Subjects can be administered subcutaneously using a pharmaceutical composition configured for subcutaneous administrations such as a prefilled syringe, microneedles, sustained-release delivery systems, and stimuli-responsive delivery systems.

The trastuzumab subcutaneous treatment solution may be supplied in a commercially available syringe infusion system such as the KORU™ FreedomEdge® (available from KORU™ Medical Systems, Chester, NY, USA).

In an embodiment, the trastuzumab subcutaneous treatment solution can be administered by using a prefilled syringe including 3 mL to 7 mL of the trastuzumab subcutaneous treatment solution.

In another embodiment, the trastuzumab subcutaneous treatment solution may be supplied in a commercially available medical device, such as an injection device, an infusion pump, an autoinjector device, a needleless device, or through subcutaneous patch delivery system, but not limited thereto. For example, the trastuzumab subcutaneous treatment solution may be supplied in a commercially available infusion pump such as the Crono S-PID Pump (available from Canes S.P.A) and described by U.S. Pat. No. 8,172,814B2. The trastuzumab subcutaneous treatment solution may be supplied in another commercially available infusion pump such as the SCIg60 Infuser (available from EMED Technologies Corporation) and described by U.S. Pat. No. 9,808,576B2 and US Patent publication No. 20130138075.

In an embodiment, the trastuzumab subcutaneous treatment solution can be administered by using an infusion pump including 10 mL to 15 mL of the trastuzumab subcutaneous treatment solution.

In other embodiments, the trastuzumab subcutaneous treatment solution may be supplied in a commercially available On-body injector such as Large Volume Injector (LVI-V), LVI-P, LVI-U, Dual Cartridge Injector (DCI), Auto-Reconstitution Injector (ART) (available from Sonceboz S.A.) and described by European Patent No. 3354303B1. The trastuzumab subcutaneous treatment solution may be supplied in a commercially available On-body injector such as SmartDose (available from West Pharmaceutical Services) and described by WIPO Patent Publication No. 2018222521A1 and WIPO Patent Publication No. 2019032395A1. The trastuzumab subcutaneous treatment solution may be supplied in a commercially available On-body injector such as enFuse® On-Body Infusor (available from Enable Injections Inc.) and described by US Patent Publication No. 20180161497A1 and US Patent Publication No. 20210353222A1.

In other embodiments, the trastuzumab subcutaneous treatment solution may be supplied in a commercially available autoinjector such as an autoinjector device available from West Pharmaceutical Services and described by U.S. Pat. No. 8,048,029B2.

It will be understood that the amount of the compound administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

### EXAMPLES

### Sample Preparation

High-concentrated trastuzumab is made from IV trastuzumab that has been reprocessed via ultrafiltration/diafiltration (UF/DF) and filtration. Concentration and buffer exchange are the key process steps in high-concentrated trastuzumab manufacture. Centrifugal filtration and tangential flow filtration (TFF) are two approaches to achieve buffer exchange and concentrate for mAbs. In early small-scale development, limited sample availability required that the buffer exchange be carried out via dialysis tubing or a dialysis cassette with 20 kDa cut-off porous membrane. Samples of high-concentrated trastuzumab were then concentrated by repeatedly centrifuging through a 30 kDa cut-off centrifugal filter with a spin speed of 4200 rpm at 4° C. for 20 min to achieve the target concentration. The centrifuge used was an Eppendorf 5804R with S-4-72 swing rotor.

In large-scale laboratory development, high-concentrated trastuzumab sample preparation includes using TFF to concentrate the mAb and exchange the buffer. TFF process was carried out using a Merck Millipore Labscale TFF System® with Pellicon® 3 with an Ultracel® 30 kDa membrane, D cassette. Trastuzumab was diafiltrated with test formulation buffers and concentrated to the target concentration.

High-concentrated pertuzumab is made from pertuzumab that has been processed via chromatography, ultrafiltration/diafiltration (UF/DF) and filtration. Concentration and buffer exchange are the key process steps in pertuzumab manufacture. Centrifugal filtration and tangential flow filtration (TFF) are two approaches to achieve buffer exchange and concentrate for mAbs. In early small-scale development, limited sample availability required that the buffer exchange be carried out via dialysis tubing or a dialysis cassette with 20 kDa cut-off porous membrane. Samples of pertuzumab were then concentrated by repeatedly centrifuging through a 30 kDa cut-off centrifugal filter with a spin speed of 4200 rpm at 4° C. for 20 min to achieve the target concentration. The centrifuge used was an Eppendorf 5804R with S-4-72 swing rotor.

In large-scale laboratory development, pertuzumab sample preparation includes using TFF to concentrate the mAb and exchange the buffer. TFF process was carried out using a Merck Milipore Labscale TFF System® with Pellicon® 3 with an Ultracel® 30 kDa membrane, D cassette. Pertuzumab was diafiltrated with test formulation buffers and concentrated to the target concentration.

### Methods for Testing Stability

### Turbidity

Turbidity is the detection of visible/subvisible particles, thus testing both the quality and stability of the formulation. The sample was inverted gently 10 times before loading onto a 96-microwell plate (Cat No. 269620). 200 μL of the sample was loaded into each well in triplicate. The samples were measured three times by SpectraMax® iD3 Multi-Mode Microplate reader. The readings were recorded by average triplicate absorbance.

### Purity, Aggregate, and Cleavages

The purity, aggregation, and cleavages of the antibodies were measured by Size Exclusion High Performance Liquid Chromatography (SEC-HPLC) using HPLC system HPLC-A6002 (Agilent 1260 Infinity II). Column (TOSOH TSK-gel G3000 SWx1, 7.8×300 mm, PN: 0008541) was provided.

### Charged Variants

The charged variants of the antibodies were measured by Cation Exchange High Performance Liquid Chromatography (CIX-HPLC) using HPLC system HPLC-A6002 (Agilent 1260 Infinity II series). Column (TOSOH TSK-gel CM-STAT, 4.6×100 mm, 7 um, CN: 0021966, Lot:082GA00017G) was provided. Main component (main peak), acidic variant, and basic variants of the antibodies were detected from the sample.

### Colloidal Stability, Aggregation and Particle Size

The colloidal stability, aggregation and particle size were measured by Dynamic Light Scattering (DLS) using Dynamic Light Scattering DynaPro® Plate Reader III (Wyatt Technology Corp., CA) equipped with 823.3 nm laser wavelength and 1500 scattering angle.

Dynamic Light Scattering was used to measure the size distribution profile of vesicles in solution. Light passing through small particles in solution would scatter. Samples were gently inverted 10 times before loading onto an Auroa 384 microplate (Wyatt PN: P8806-38401). Initial temperature and end temperature are set at 25° C. and 75° C. respectively for Tₒₙₛₑₜ or T_{agg}. Each well of the microplate was read 5 times and an average of measurements was recorded for each well. The measurement of the scattering constant and the concentration of the sample were plotted on Y-axis and X-axis respectively. A positive slope indicates light passes through the space between small particles and little aggregation in the concentrated sample. A negative slope indicates a larger particle size due to aggregation in the concentrated sample.

### Protein Content Assay

Protein concentration was measured by a protein content assay using Spectrophotometer Jasco v-730. The detection wavelength was set at 280 nm. Sample was diluted to an appropriate concentration including OD 0.4-0.8. 110 μL of the Sample was loaded into a cuvette and measured at 280 nm. Average absorbance was recorded and the concentration of the protein (antibody) was calculated.

Specifically, the measuring method of the protein concentration included: turning on the spectrophotometer and the UV lamp at least 20 minutes before use.

280 nm was the detection wavelength. A 1 cm quartz cuvette was washed with ultrapure water and dried. The cuvette was rinsed with formulation basal buffer. The formulation basal buffer was added into the cuvette. The cuvette was inserted into the spectrophotometer and recorded as blank. The formulation basal buffer was discarded and the cuvette was rinsed with testing sample for equilibrium. The testing sample was discarded from the cuvette.

Testing sample was loaded into the cuvette and measured. The sample was discarded and testing sample was again loaded to measure the samples in duplicate. The cuvette was flushed with formulation basal buffer and the step of taking a blank measurement to the step of measuring the test samples was repeated for the next testing samples.

After completing taking measurements, the cuvette was washed with ultrapure water and dried. The absorbance of each duplicated samples was averaged. The concentration was calculated by the equation of Beer's law.

### Binding Activity

### HER2-ECD Binding ELISA

Binding activity of the antibody was measured by a HER2-ECD binding ELISA assay.

### FcγRIII Binding Assay

FcγRIII binding assay is a binding kinetics method measuring the interaction between trastuzumab and its receptor by Bio-Layer Interference (BLI) technology. The measurement is conducted by using ForteBio OctetRed96 (Pall, equipment no. BAZ-20002).

### Stability Studies

### Stability Studies Example 1: Comparison of the Stability Depending on Buffer Solution

Regarding the liquid pharmaceutical formulation used in Stability Studies Example 1, each buffer solution was prepared so as to be adapted for the corresponding pH and concentration, added with an antibody, thus yielding the samples set forth in Table 1, Table 2, and Table 3 below. The specific content of each component is described in Table 1, Table 2, and Table 3 below. The total volume was, for example, 5 mL.

TABLE 1

| | | | | | | | | | | | | | | FcγRIII |
|---|---|---|---|---|---|---|---|---|---|---|---|---|---|---|
| | | Trastu- | | | | | | | | | | | | binding |
| | | zumab | | | | | Size Exclusion-HPLC | | | Ion Exchange-HPLC | | | Tur- | relative |
| Example | | Conc. | | Temp. | Storage | | HMW | Monomer | LMW | Acidic | Main | Basic | bidity | potency |
| No. | Buffer | (mg/ml) | pH | (° C.) | time | | (%) | (%) | (%) | variant(%) | Peak (%) | variant(%) | (A350) | (%) |
| Example | 20 mM | 120 | 6.0 | 4 | Initial | | 0.97 | 99.03 | 0.0 | 32.02 | 58.71 | 9.28 | 0.266 | 93.9 |
| 1 | L-Histidine/ | | | | 3 | days | 1.29 | 98.71 | 0.0 | 31.85 | 59.34 | 8.81 | 0.295 | 107.4 |
| | L-Histidine | | | | 1 | wk | 1.15 | 98.85 | 0.0 | 33.43 | 55.06 | 11.52 | 0.266 | — |
| | HCl | | | | 2 | wks | 1.14 | 98.86 | 0.0 | 32.81 | 57.85 | 9.34 | 0.27 | — |
| | | | | | 4 | wks | 1.42 | 98.58 | 0.0 | 28.98 | 60.00 | 11.02 | 0.285 | — |

| | | | | | | | | | |
|---|---|---|---|---|---|---|---|---|---|
| 40 | Initial | 0.97 | 99.03 | 0.0 | 31.65 | 58.83 | 9.52 | 0.278 | 93.9 |

| | | | | | | | | | |
|---|---|---|---|---|---|---|---|---|---|
| 3 | days | 1.74 | 98.26 | 0.0 | 38.14 | 51.03 | 10.83 | 0.3 | 97.8 |
| 1 | wk | 1.91 | 97.83 | 0.26 | 46.79 | 46.79 | 17.00 | 0.308 | — |
| 2 | wks | 2.41 | 97.33 | 0.26 | 55.66 | 26.01 | 18.34 | 0.354 | — |
| 4 | wks | 3.25 | 94.57 | 2.18 | 63.23 | 16.70 | 20.07 | 0.397 | — |

TABLE 2

| | | | | | | | | | | | | | | FcγRIII |
|---|---|---|---|---|---|---|---|---|---|---|---|---|---|---|
| | | Trastu- | | | | | | | | | | | | binding |
| | | zumab | | | | | Size Exclusion-HPLC | | | Ion Exchange-HPLC | | | Tur- | relative |
| Example | | Conc. | | Temp. | Storage | | HMW | Monomer | LMW | Acidic | Main | Basic | bidity | potency |
| No. | Buffer | (mg/ml) | pH | (° C.) | time | | (%) | (%) | (%) | variant(%) | Peak (%) | variant(%) | (A350) | (%) |
| Example | 20 mM | 120 | 5.5 | 4 | Initial | | 1.27 | 98.73 | 0.0 | 27.70 | 64.58 | 7.63 | 0.309 | 101.7 |
| 2 | Citric | | | | 3 | days | 1.43 | 98.57 | 0.0 | 26.70 | 62.70 | 10.60 | 0.333 | 90.6 |
| | Acid/ | | | | 1 | wk | 1.32 | 98.68 | 0.0 | 29.80 | 61.04 | 9.17 | 0.32 | — |
| | Sodium | | | | 2 | wks | 1.51 | 98.50 | 0.0 | 28.62 | 63.53 | 7.85 | 0.31 | — |
| | Citrate | | | | 4 | wks | 1.74 | 98.26 | 0.0 | 25.98 | 61.46 | 12.56 | 0.328 | — |

| | | | | | | | | | |
|---|---|---|---|---|---|---|---|---|---|
| 40 | Initial | 1.27 | 98.73 | 0.0 | 28.07 | 64.15 | 7.77 | 0.329 | 101.7 |

| | | | | | | | | | |
|---|---|---|---|---|---|---|---|---|---|
| 3 | days | 2.55 | 97.65 | 0.0 | 30.28 | 52.79 | 16.93 | 0.326 | 90.0 |
| 1 | wk | 2.53 | 97.24 | 0.24 | 35.34 | 45.35 | 19.31 | 0.323 | — |
| 2 | wks | 2.91 | 96.67 | 0.41 | 39.75 | 34.75 | 25.49 | 0.329 | — |
| 4 | wks | 3.56 | 94.63 | 1.81 | 40.16 | 25.35 | 34.50 | 0.332 | — |

TABLE 3

| | | | | | | | | | | | | | | FcγRIII |
|---|---|---|---|---|---|---|---|---|---|---|---|---|---|---|
| | | Trastu- | | | | | | | | | | | | binding |
| | | zumab | | | | | Size Exclusion-HPLC | | | Ion Exchange-HPLC | | | Tur- | relative |
| Example | | Conc. | | Temp. | Storage | | HMW | Monomer | LMW | Acidic | Main | Basic | bidity | potency |
| No. | Buffer | (mg/ml) | pH | (° C.) | time | | (%) | (%) | (%) | variant(%) | Peak (%) | variant(%) | (A350) | (%) |
| Example | 20 mM | 120 | 5.5 | 4 | Initial | | 0.93 | 99.08 | 0.0 | 28.96 | 63.15 | 7.89 | 0.231 | 103.4 |
| 3 | Acetic | | | | 3 | days | 1.22 | 98.78 | 0.0 | 30.20 | 57.83 | 11.97 | 0.24 | 94.2 |
| | Acid/ | | | | 1 | wk | 1.05 | 98.95 | 0.0 | 29.84 | 60.56 | 9.60 | 0.238 | — |
| | Sodium | | | | 2 | wks | 1.22 | 98.78 | 0.0 | 29.78 | 61.57 | 8.65 | 0.238 | — |
| | acetate | | | | 4 | wks | 1.36 | 95.64 | 0.0 | 26.60 | 62.10 | 11.31 | 0.24 | — |

| | | | | | | | | | |
|---|---|---|---|---|---|---|---|---|---|
| 40 | Initial | 0.93 | 99.08 | 0.0 | 28.51 | 63.65 | 7.83 | 0.24 | 103.4 |

| | | | | | | | | | |
|---|---|---|---|---|---|---|---|---|---|
| 3 | days | 1.85 | 98.15 | 0.0 | 32.25 | 50.95 | 16.80 | 0.241 | 96.0 |
| 1 | wk | 2.01 | 97.78 | 0.21 | 36.41 | 44.24 | 19.35 | 0.241 | — |
| 2 | wks | 2.57 | 97.12 | 0.31 | 40.65 | 34.25 | 25.12 | 0.244 | — |
| 4 | wks | 3.11 | 94.99 | 1.90 | 40.92 | 26.13 | 32.95 | 0.252 | — |

The liquid pharmaceutical formulations prepared in Examples 1 to 3 were measured for stability after 0 days (marked as Initial), 3 days, 1 week, 2 weeks, and 4 weeks at a temperature of 5° C.±3° C. or 40° C.±2° C. Example 1 represents a Histidine buffer system using 20 mM L-Histidine/L-Histidine HCl solution. Example 2 represents a citric acid system using 20 mM Citric acid/Sodium Citrate solution. Example 3 represents an Acetic acid system using 20 mM Acetic Acid/Sodium acetate solution. The results are shown in Tables 1 to 3 above.

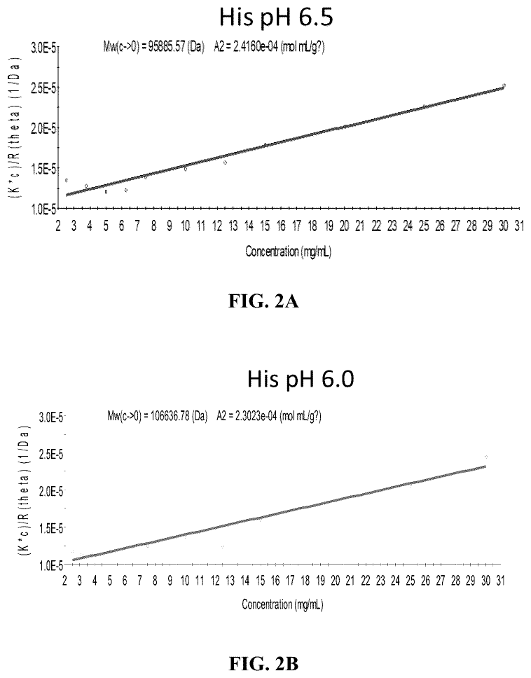

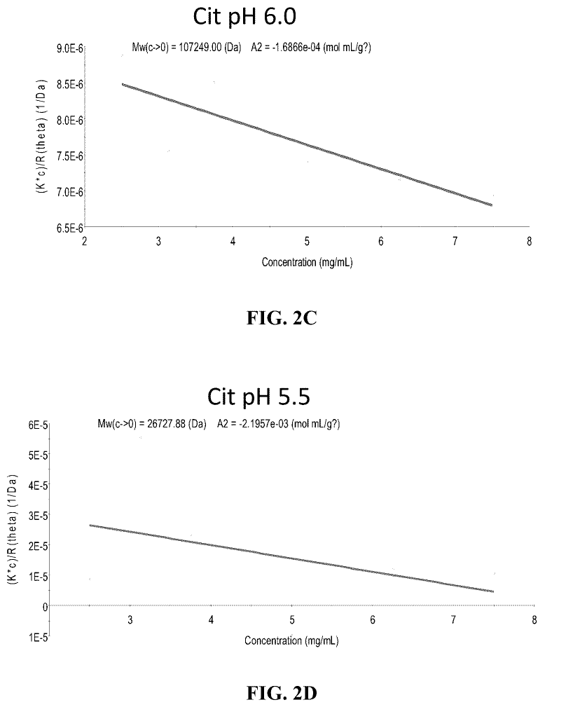

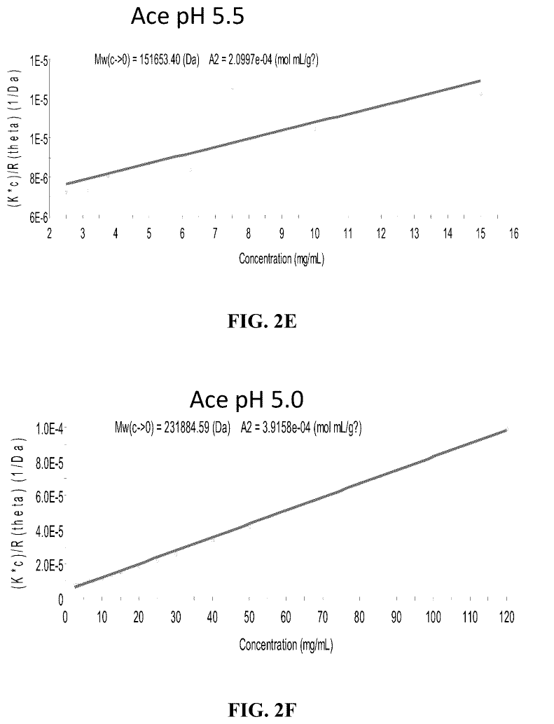

FIG. 2 shows the colloidal stability of trastuzumab in different buffers. Using DLS, scattering constant v. trastuzumab concentration were plotted. FIG. 2A shows the colloidal stability of trastuzumab in Histidine buffer at pH 6.5. FIG. 2B shows the colloidal stability of trastuzumab in Histidine buffer at pH 6.0. FIG. 2C shows the colloidal stability of trastuzumab in Citric acid buffer at pH 6.0. FIG. 2D shows the colloidal stability of trastuzumab in Citric acid buffer at pH 5.5. FIG. 2E shows the colloidal stability of trastuzumab in Acetic acid buffer at pH 5.5. FIG. 2F shows the colloidal stability of trastuzumab in Acetic acid buffer at pH 5.0. Trastuzumab colloidal stability in Histidine and Acetic acid buffers show a positive slope. Trastuzumab colloidal stability in Citric acid buffer shows a negative slope. In an embodiment, Histidine buffer, Acetic acid buffer or Citric acid buffer may be used as a buffer in the liquid pharmaceutical composition.

### Stability Studies Example 2: Composition of Stability Depending on pH of Acetic Acid Buffer

Regarding the liquid pharmaceutical formulations used in Stability Studies Example 2, each buffer solution was prepared so as to be adapted for the corresponding pH and antibody concentration, thus yielding the samples set forth in Table 4 below. The concentration of each component is described in Table 4 below. The total volume was, for example, 5 mL

TABLE 4

| | Trastu- | | | | | | | | | | |
|---|---|---|---|---|---|---|---|---|---|---|---|
| | zumab | | | | | Size Exclusion-HPLC | | | Ion Exchange-HPLC | | |
| | Conc. | | Temp. | Storage | | HMW | Monomer | LMW | Acidic | Main | Basic |
| Buffer | (mg/ml) | pH | (° C.) | time | | (%) | (%) | (%) | variant (%) | Peak (%) | variant (%) |
| 20 mM | 120 | 5.4 | 4 | Initial | | 0.97 | 99.03 | 0.0 | 23.70 | 64.86 | 11.45 |
| Acetic | | | | 1 | wk | 1.10 | 98.91 | 0.0 | 25.83 | 64.71 | 9.47 |
| Acid/ | | | | 2 | wks | 1.14 | 98.86 | 0.0 | 29.97 | 59.70 | 10.33 |
| Sodium | | | | 3 | wks | 1.16 | 98.84 | 0.0 | 26.93 | 57.06 | 16.01 |
| acetate | | | | 4 | wks | 1.22 | 98.78 | 0.0 | 27.37 | 60.83 | 11.80 |

| | | | | | | | |
|---|---|---|---|---|---|---|---|
| 40 | Initial | 0.95 | 99.05 | 0.0 | 24.01 | 64.00 | 11.99 |

| | | | | | | | |
|---|---|---|---|---|---|---|---|
| 1 | wk | 01.89 | 97.95 | 0.17 | 27.09 | 49.70 | 23.21 |
| 2 | wks | 2.25 | 97.47 | 0.28 | 35.39 | 35.79 | 28.82 |
| 3 | wks | 2.21 | 97.49 | 0.29 | 33.06 | 31.43 | 35.51 |
| 4 | wks | 3.04 | 96.57 | 0.39 | 37.27 | 28.64 | 34.07 |

| | | | | | | | | |
|---|---|---|---|---|---|---|---|---|
| 5.0 | 4 | Initial | 1.16 | 98.84 | 0.0 | 24.28 | 65.59 | 10.13 |

| | | | | | | | |
|---|---|---|---|---|---|---|---|
| 1 | wk | 1.22 | 97.78 | 0.0 | 25.01 | 65.60 | 9.39 |
| 2 | wks | 1.27 | 98.73 | 0.0 | 29.70 | 59.82 | 10.49 |
| 3 | wks | 1.24 | 98.76 | 0.0 | 26.55 | 59.44 | 14.01 |
| 4 | wks | 1.43 | 98.57 | 0.0 | 27.19 | 61.43 | 11.38 |

| | | | | | | | |
|---|---|---|---|---|---|---|---|
| 40 | Initial | 1.15 | 98.85 | 0.0 | 24.59 | 63.44 | 11.97 |

| | | | | | | | |
|---|---|---|---|---|---|---|---|
| 1 | wk | 2.21 | 97.56 | 0.23 | 26.93 | 47.36 | 25.72 |
| 2 | wks | 2.60 | 97.03 | 0.37 | 33.66 | 33.80 | 32.54 |
| 3 | wks | 2.31 | 97.24 | 0.45 | 30.29 | 29.53 | 40.18 |
| 4 | wks | 2.94 | 96.43 | 0.63 | 33.03 | 26.73 | 40.24 |

| | | | | | | | | |
|---|---|---|---|---|---|---|---|---|
| 4.6 | 4 | Initial | 0.92 | 99.08 | 0.0 | 23.73 | 60.64 | 15.63 |

| | | | | | | | |
|---|---|---|---|---|---|---|---|
| 1 | wk | 0.69 | 99.31 | 0.0 | 25.38 | 63.10 | 11.52 |
| 2 | wks | 0.69 | 99.31 | 0.0 | 31.63 | 55.64 | 12.73 |
| 3 | wks | 0.72 | 99.28 | 0.0 | 26.39 | 55.57 | 18.05 |
| 4 | wks | 0.81 | 99.19 | 0.0 | 28.38 | 58.41 | 13.21 |

| | | | | | | | |
|---|---|---|---|---|---|---|---|
| 40 | Initial | 0.7 | 99.30 | 0.0 | 23.77 | 61.18 | 15.06 |

| | | | | | | | |
|---|---|---|---|---|---|---|---|
| 1 | wk | 0.93 | 98.89 | 0.18 | 25.48 | 46.79 | 27.74 |
| 2 | wks | 1.21 | 98.45 | 0.34 | 33.25 | 31.91 | 34.83 |
| 3 | wks | 1.98 | 97.57 | 0.45 | 27.68 | 30.96 | 41.37 |
| 4 | wks | 1.40 | 98.07 | 0.54 | 26.69 | 27.84 | 45.47 |

The above formulations were measured for stability after 0 weeks (Initial), 1 week, 1 month, 2 months, and 3 months at a temperature of 40±2° C. Examples 4, 5, 6 represent formulations using 20 mM Acetic Acid/Sodium acetate solution at pH 5.4, 5.0 or 4.6 respectively. The results are shown in Table 4 above.

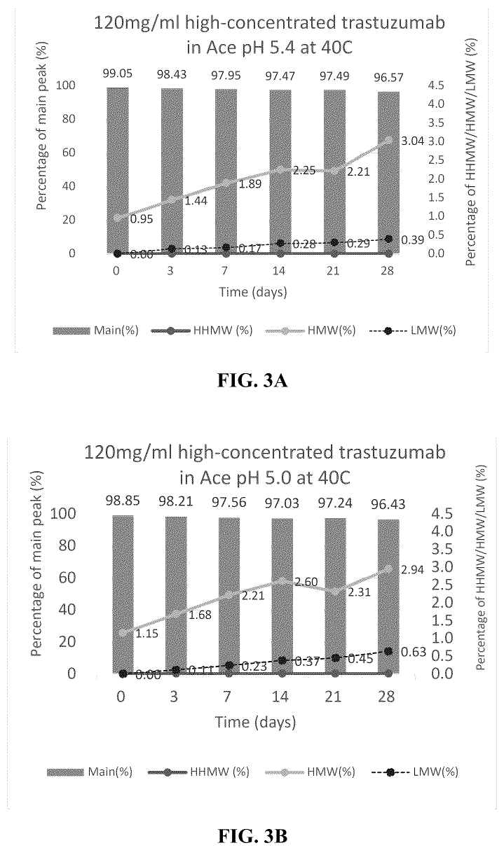

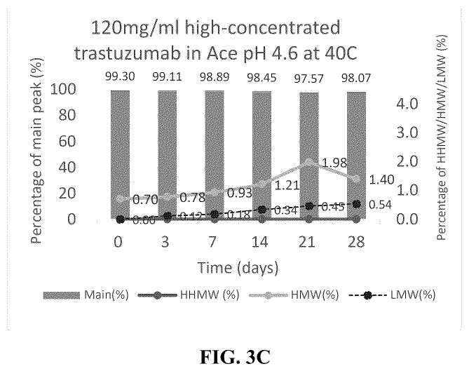

FIG. 3 shows the antibody profile as measured by SEC-HPLC. FIG. 3 shows the antibody profile of high-concentrated trastuzumab over 28 days under accelerated thermal stress of 40° C. FIG. 3A shows an antibody profile under Acetic acid at pH 5.4. FIG. 3B shows an antibody profile under Acetic acid at pH 5.0. FIG. 3C shows the antibody profile under Acetic acid at pH 4.6.

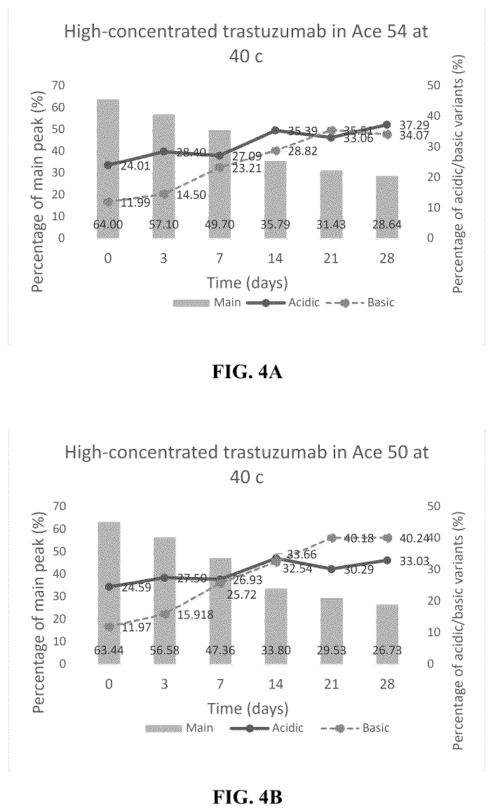

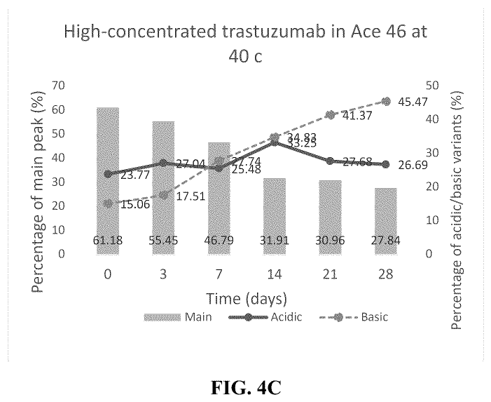

FIG. 4 shows the antibody as measured by CIX-HPLC. FIG. 4 shows the charge variants profile of the antibody over 28 days under accelerated thermal stress of 40° C. FIG. 4A shows the charge variant profile under Acetic acid at pH 5.4. FIG. 4B shows the charge variant profile under Acetic acid at pH 5.0. FIG. 4C shows the charge variant profile under Acetic acid at pH 4.6.

In an embodiment, Acetic acid may have a pH of 4.6 to 5.4.

### Stability Studies Example 3: Composition of Stability Depending on Types of Additives or pH in Acetic Acid Buffer

Regarding the liquid pharmaceutical formulations used in Stability Studies Example 3, each buffer solution was prepared so as to be adapted for the corresponding pH and antibody concentration, with addition of a tonicity agent or salt, thus yielding the samples set forth in Table 5 below. The concentration of each component is described in Table 5 below. The total volume was, for example, 5 mL.

TABLE 5

| | | Trastu- | | | | | | | | | | | | |
|---|---|---|---|---|---|---|---|---|---|---|---|---|---|---|
| | | zumab | | Organic | | | | | Size Exclusion-HPLC | | | Ion Exchange-HPLC | | |
| Example | | Conc. | | co- | Temp. | | Storage | | HMW | Monomer | LMW | Acidic | Main | Basic |
| No. | Buffer | (mg/ml) | pH | solvent | (° C.) | Additive | time | | (%) | (%) | (%) | variant(%) | Peak (%) | variant(%) |
| Example | 20 mM | 120 | 5.6 | 0.05% | 4 | 150 mM | Initial | | 1.33 | 98.67 | — | 29.09 | 59.6 | 11.31 |
| 7 | Acetic | | | PS20 | | trehalose | 1 | wk | 1.50 | 98.50 | — | 27.71 | 63.8 | 8.54 |
| | Acid/ | | | | | | 2 | wks | 1.58 | 98.43 | — | 27.08 | 62.9 | 10.00 |
| | Sodium | | | | | | 4 | wks | 1.73 | 98.27 | — | 25.82 | 63.9 | 10.33 |
| | acetate | | | | | 0.9% | Initial | | 1.31 | 98.69 | — | 31.47 | 57.8 | 10.69 |
| | | | | | | NaCl | 1 | wk | 1.70 | 98.30 | — | 27.41 | 65.8 | 6.79 |
| | | | | | | | 2 | wks | 1.94 | 98.06 | — | 29.33 | 60.1 | 10.52 |
| | | | | | | | 4 | wks | 2.02 | 97.99 | — | 25.43 | 64.3 | 10.27 |
| | | | | | 40 | 150 mM | 1 | wk | 2.85 | 97.00 | 0.15 | 30.31 | 47.5 | 22.18 |
| | | | | | | trehalose | 2 | wks | 3.23 | 96.49 | 0.28 | 38.81 | 33.7 | 27.50 |
| | | | | | | | 4 | wks | 3.82 | 95.73 | 0.44 | 69.04 | 18.3 | 12.62 |
| | | | | | | 0.9% | 1 | wk | 3.22 | 96.67 | 0.12 | 29.52 | 49.2 | 21.30 |
| | | | | | | NaCl | 2 | wks | 3.86 | 95.84 | 0.31 | 37.68 | 35.4 | 26.87 |
| | | | | | | | 4 | wks | 4.22 | 95.29 | 0.50 | 68.82 | 18.9 | 12.30 |
| Example | | | 5.2 | | 4 | 150 mM | Initial | | 1.13 | 98.87 | — | 30.57 | 56.5 | 12.94 |
| 8 | | | | | | trehalose | 1 | wk | 1.03 | 98.97 | — | 25.47 | 62.2 | 12.29 |
| | | | | | | | 2 | wks | 1.08 | 98.92 | — | 26.86 | 60.9 | 12.20 |
| | | | | | | | 4 | wks | 1.09 | 98.91 | — | 24.28 | 63.1 | 12.63 |

| | 0.9% | Initial | | 1.14 | 98.86 | — | 29.30 | 57.7 | 12.95 |
|---|---|---|---|---|---|---|---|---|---|
| | NaCl | 1 | wk | 1.25 | 98.75 | — | 27.37 | 61.6 | 11.01 |
| | | 2 | wks | 1.25 | 98.75 | — | 32.89 | 59.1 | 8.06 |
| | | 4 | wks | 1.36 | 98.64 | — | 24.60 | 62.8 | 12.55 |
| 40 | 150 mM | 1 | wk | 1.50 | 98.33 | 0.18 | 26.43 | 47.2 | 26.32 |
| | trehalose | 2 | wks | 1.76 | 98.00 | 0.25 | 34.00 | 37.8 | 28.24 |
| | | 4 | wks | 1.97 | 97.62 | 0.41 | 63.04 | 20.8 | 16.17 |
| | 0.9% | 1 | wk | 2.06 | 97.80 | 0.15 | 24.84 | 49.1 | 26.03 |
| | NaCl | 2 | wks | 2.46 | 97.26 | 0.29 | 31.55 | 38.0 | 30.49 |
| | | 4 | wks | 2.56 | 96.91 | 0.53 | 61.73 | 21.3 | 16.98 |

The above formulations were measured for stability after 0 weeks (Initial), 1 week, 2 weeks, and 4 weeks at a temperature of 5° C.±3° C. or 40° C.±2° C. Examples 7, 8 represent formulations including 0.05% PS20, 150 mM trehalose or 0.9% NaCl, and 20 mM Acetic Acid/Sodium acetate solution at pH 5.6 or 5.2 respectively. The concentration of the antibody is 120 mg/mL The results are shown in Table 5 above.

In an embodiment, the formulation may include Acetic Acid/Sodium acetate solution at pH between 5.6 to 5.2. In an embodiment, the formulation may include tonicity agents or salts. The tonicity agent may be trehalose, but not limited thereto. The salt may include sodium chloride (NaCl), but not limited thereto.

### Stability Studies Example 4: Composition of Stability Depending on Types of Tonicity Agents and Buffers

Regarding the liquid pharmaceutical formulations used in Stability Studies Example 4, each buffer solution was prepared so as to be adapted for the corresponding buffers, tonicity agents, and antibody concentration, with addition of salt, and further addition of amino acids, thus yielding the samples set forth in Tables 6 to 13 below. The concentration of each component is described in Tables 6 to 13 below. The total volume was, for example, 5 mL.

TABLE 6

| | Trastu- | | | Treha- | | | | | |
|---|---|---|---|---|---|---|---|---|---|
| Sample | zumab | | | lose | Met | Arg | NaCl | | |
| Test | Conc. | Temp. | | Conc. | Conc. | Conc. | Conc. | Size Exclusion-HPLC | |
| No. | (mg/ml) | (° C.) | Buffer | (mM) | (mM) | (mM) | (mM) | HMW (%) | |
| 1 | 120 | 40 | 20 mM | 20 | 20 | 0 | 0 | Initial | 1.19 |
| 2 | | | His | 20 | 20 | 50 | 0 | Initial | 2.16 |
| 3 | | | buffer | 300 | 20 | 0 | 0 | Initial | 1.08 |
| 4 | | | pH6.0 | 20 | 20 | 0 | 150 | Initial | 1.13 |
| 5 | | | | 20 | 20 | 50 | 150 | Initial | 2.14 |
| 6 | | | | 20 | 0 | 0 | 0 | Initial | 1.14 |
| 7 | | | | 300 | 0 | 0 | 0 | Initial | 1.18 |

| Sample | | | | | | | | | | |
|---|---|---|---|---|---|---|---|---|---|---|
| Test | Size Exclusion-HPLC | | | | | | | | | |
| No. | HMW (%) | | Monomer (%) | | | | LMW (%) | | | |
| 1 | day 4 | 4.71 | Initial | 98.65 | day 4 | 91.63 | Initial | 0.16 | day 4 | 3.67 |
| 2 | day 4 | 100 | Initial | 97.73 | day 4 | 0 | Initial | 0.11 | day 4 | 0 |
| 3 | day 4 | 6.88 | Initial | 98.87 | day 4 | 89.83 | Initial | 0.05 | day 4 | 3.29 |
| 4 | day 4 | 3.88 | Initial | 98.8 | day 4 | 92.44 | Initial | 0.07 | day 4 | 3.67 |
| 5 | day 4 | 100 | Initial | 97.77 | day 4 | 0 | Initial | 0.08 | day 4 | 0 |
| 6 | day 4 | 9.14 | Initial | 98.8 | day 4 | 87.46 | Initial | 0.06 | day 4 | 3.39 |
| 7 | day 4 | 7.94 | Initial | 98.82 | day 4 | 88.89 | Initial | 0 | day 4 | 3.16 |

The above formulations were measured for stability after 0 days (Initial), and 4 days at a temperature of 40° C.±2° C. Formulations represented by samples 1 to 7 include 20 mM Histidine buffer at pH 6.0 and 20 mM or 300 mM Trehalose. The concentration of the antibody is 120 mg/mL The results are shown in Table 6 above.

In an embodiment, the formulation may include Histidine buffer, Trehalose, Methionine or NaCl, or a combination thereof, but not limited thereto.

TABLE 7

| | Trastu- | | | Man- | | | | | |
|---|---|---|---|---|---|---|---|---|---|
| Sample | zumab | | | nitol | Met | Arg | NaCl | | |
| Test | Conc. | Temp. | | Conc. | Conc. | Conc. | Conc. | Size Exclusion-HPLC | |
| No. | (mg/ml) | (° C.) | Buffer | (mM) | (mM) | (mM) | (mM) | HMW (%) | |
| 8 | 120 | 40 | 20 mM | 20 | 20 | 0 | 0 | Initial | 1.09 |
| 9 | | | His | 20 | 20 | 50 | 0 | Initial | 1.08 |
| 10 | | | buffer | 300 | 20 | 0 | 0 | Initial | 0.94 |
| 11 | | | pH6.0 | 20 | 20 | 0 | 150 | Initial | 1.13 |
| 12 | | | | 20 | 20 | 50 | 150 | Initial | 1.89 |
| 13 | | | | 20 | 0 | 0 | 0 | Initial | 1.12 |
| 14 | | | | 300 | 0 | 0 | 0 | Initial | 0.94 |

| Sample | | | | | | | | | | |
|---|---|---|---|---|---|---|---|---|---|---|
| Test | Size Exclusion-HPLC | | | | | | | | | |
| No. | HMW (%) | | Monomer (%) | | | | LMW (%) | | | |
| 8 | day 4 | 9.18 | Initial | 98.82 | day 4 | 90.38 | Initial | 0.09 | day 4 | 0.45 |
| 9 | day 4 | 100 | Initial | 98.85 | day 4 | 0 | Initial | 0.07 | day 4 | 0 |
| 10 | day 4 | 9.97 | Initial | 99.06 | day 4 | 87.05 | Initial | 0 | day 4 | 2.98 |
| 11 | day 4 | 17.1 | Initial | 98.81 | day 4 | 82.49 | Initial | 0.05 | day 4 | 0.42 |
| 12 | day 4 | 90 | Initial | 98.11 | day 4 | 10 | Initial | 0 | day 4 | 0 |
| 13 | day 4 | 9.92 | Initial | 98.22 | day 4 | 89.65 | Initial | 0 | day 4 | 0.43 |
| 14 | day 4 | 17.12 | Initial | 99.06 | day 4 | 80.16 | Initial | 0 | day 4 | 2.73 |

The above formulations were measured for stability after 0 days (Initial), and 4 days at a temperature of 40° C.±2° C. Formulations represented by samples 8 to 14 include 20 mM Histidine buffer at pH 6.0 and 20 mM or 300 mM Mannitol. The concentration of the antibody is 120 mg/mL The results are shown in Table 7 above. In an embodiment, the formulation may include Histidine buffer, Mannitol, Methionine or NaCl, or a combination thereof, but not limited thereto.

TABLE 8

| | Trastu- | | | Sor- | | | | | |
|---|---|---|---|---|---|---|---|---|---|
| Sample | zumab | | | bitol | Met | Arg | NaCl | | |
| Test | Conc. | Temp. | | Conc. | Conc. | Conc. | Conc. | Size Exclusion-HPLC | |
| No. | (mg/ml) | (° C.) | Buffer | (mM) | (mM) | (mM) | (mM) | HMW (%) | |
| 15 | 120 | 40 | 20 mM | 20 | 20 | 0 | 0 | Initial | 0.88 |
| 16 | | | His | 20 | 20 | 50 | 0 | Initial | 1.94 |
| 17 | | | buffer | 300 | 20 | 0 | 0 | Initial | 1.17 |
| 18 | | | pH6.0 | 20 | 20 | 0 | 150 | Initial | 1.16 |
| 19 | | | | 20 | 20 | 50 | 150 | Initial | 1.94 |
| 20 | | | | 20 | 0 | 0 | 0 | Initial | 1.17 |
| 21 | | | | 300 | 0 | 0 | 0 | Initial | 1.19 |

| Sample | | | | | | | | | | |
|---|---|---|---|---|---|---|---|---|---|---|
| Test | Size Exclusion-HPLC | | | | | | | | | |
| No. | HMW (%) | | Monomer (%) | | | | LMW (%) | | | |
| 15 | day 4 | 11.04 | Initial | 99.13 | day 4 | 85.68 | Initial | 0 | day 4 | 3.27 |
| 16 | day 4 | 100 | Initial | 98.06 | day 4 | 0 | Initial | 0 | day 4 | 0 |
| 17 | day 4 | 17.19 | Initial | 98.83 | day 4 | 79.87 | Initial | 0 | day 4 | 2.93 |
| 18 | day 4 | 14.86 | Initial | 98.84 | day 4 | 81.98 | Initial | 0 | day 4 | 3.17 |
| 19 | day 4 | 100 | Initial | 98.06 | day 4 | 0 | Initial | 0 | day 4 | 0 |
| 20 | day 4 | 14.43 | Initial | 98.83 | day 4 | 82.52 | Initial | 0 | day 4 | 3.06 |
| 21 | day 4 | 20.36 | Initial | 98.81 | day 4 | 76.14 | Initial | 0 | day 4 | 3.49 |

The above formulations were measured for stability after 0 days (Initial), and 4 days at a temperature of 40° C.±2° C. Formulations represented by samples 15 to 21 include 20 mM Histidine buffer at pH 6.0 and 20 mM or 300 mM Sorbitol. The concentration of the antibody is 120 mg/mL The results are shown in Table 8 above. In an embodiment, the formulation may include Histidine buffer, Sorbitol, Methionine or NaCl, or a combination thereof, but not limited thereto.

TABLE 9

| | Trastu- | | | | | | | | |
|---|---|---|---|---|---|---|---|---|---|
| Sample | zumab | | | Sucrose | Met | Arg | NaCl | | |
| Test | Conc. | Temp. | | Conc. | Conc. | Conc. | Conc. | Size Exclusion-HPLC | |
| No. | (mg/ml) | (° C.) | Buffer | (mM) | (mM) | (mM) | (mM) | HMW (%) | |
| 22 | 120 | 40 | 20 mM | 20 | 20 | 0 | 0 | Initial | 1.12 |
| 23 | | | His | 20 | 20 | 50 | 0 | Initial | 2.13 |
| 24 | | | buffer | 300 | 20 | 0 | 0 | Initial | 1.12 |
| 25 | | | pH6.0 | 20 | 20 | 0 | 150 | Initial | 1.16 |
| 26 | | | | 20 | 20 | 50 | 150 | Initial | 2.14 |
| 27 | | | | 20 | 0 | 0 | 0 | Initial | 1.16 |
| 28 | | | | 300 | 0 | 0 | 0 | Initial | 1.21 |

| Sample | | | | | | | | | | |
|---|---|---|---|---|---|---|---|---|---|---|
| Test | Size Exclusion-HPLC | | | | | | | | | |
| No. | HMW (%) | | Monomer (%) | | | | LMW (%) | | | |
| 22 | day 4 | 10.31 | Initial | 98.79 | day 4 | 86.33 | Initial | 0.09 | day 4 | 3.37 |
| 23 | day 4 | 100 | Initial | 97.8 | day 4 | 0 | Initial | 0.07 | day 4 | 0 |
| 24 | day 4 | 7.54 | Initial | 98.88 | day 4 | 92.08 | Initial | 0 | day 4 | 0.38 |
| 25 | day 4 | 13.54 | Initial | 98.79 | day 4 | 83.26 | Initial | 0.05 | day 4 | 3.2 |
| 26 | day 4 | 100 | Initial | 97.85 | day 4 | 0 | Initial | 0 | day 4 | 0 |
| 27 | day 4 | 14.02 | Initial | 98.84 | day 4 | 82.82 | Initial | 0 | day 4 | 3.15 |
| 28 | day 4 | 10.27 | Initial | 98.79 | day 4 | 74.94 | Initial | 0 | day 4 | 14.79 |

It is worth noting that the formulation including sucrose and methionine shows a higher monomer (%) at 92.08% after 4-days exposure at 40° C.±2° C. over the formulation including sorbitol and methionine at 79.87%.

The above formulations were measured for stability after 0 days (Initial), and 4 days at a temperature of 40° C.±2° C. Formulations represented by samples 22 to 28 include 20 mM Histidine buffer at pH 6.0 and 20 mM or 300 mM Sucrose. The concentration of the antibody is 120 mg/mL The results are shown in Table 9 above. In an embodiment, the formulation may include Histidine buffer, Sucrose, Methionine or NaCl, or a combination thereof, but not limited thereto.

TABLE 10

| | Trastu- | | | Treha- | | | | | |
|---|---|---|---|---|---|---|---|---|---|
| Sample | zumab | | | lose | Met | Arg | NaCl | | |
| Test | Conc. | Temp. | | Conc. | Conc. | Conc. | Conc. | Size Exclusion-HPLC | |
| No. | (mg/ml) | (° C.) | Buffer | (mM) | (mM) | (mM) | (mM) | HMW (%) | |
| 29 | 120 | 40 | 20 mM | 20 | 20 | 0 | 0 | Initial | 0.7 |
| 30 | | | Ace | 20 | 20 | 50 | 0 | Initial | 1.87 |
| 31 | | | buffer | 300 | 20 | 0 | 0 | Initial | 0.69 |
| 32 | | | pH5.0 | 20 | 20 | 0 | 150 | Initial | 0.84 |
| 33 | | | | 20 | 20 | 50 | 150 | Initial | 1.94 |
| 34 | | | | 20 | 0 | 0 | 0 | Initial | 0.76 |
| 35 | | | | 300 | 0 | 0 | 0 | Initial | 0.73 |

| Sample | | | | | | | | | | |
|---|---|---|---|---|---|---|---|---|---|---|
| Test | Size Exclusion-HPLC | | | | | | | | | |
| No. | HMW (%) | | Monomer (%) | | | | LMW (%) | | | |
| 29 | day 4 | 10.93 | Initial | 99.3 | day 4 | 86.13 | Initial | 0 | day 4 | 2.93 |
| 30 | day 4 | 100 | Initial | 98.13 | day 4 | 0 | Initial | 0 | day 4 | 0 |
| 31 | day 4 | 6.52 | Initial | 99.31 | day 4 | 90.56 | Initial | 0 | day 4 | 2.93 |
| 32 | day 4 | 38.28 | Initial | 99.16 | day 4 | 59.65 | Initial | 0 | day 4 | 2.06 |
| 33 | day 4 | 100 | Initial | 98.06 | day 4 | 0 | Initial | 0 | day 4 | 0 |
| 34 | day 4 | 12.08 | Initial | 99.24 | day 4 | 85.45 | Initial | 0 | day 4 | 2.47 |
| 35 | day 4 | 6.96 | Initial | 99.27 | day 4 | 90.46 | Initial | 0 | day 4 | 2.58 |

The above formulations were measured for stability after 0 days (Initial), and 4 days at a temperature of 40° C.±2° C. Formulations represented by samples 29 to 35 include 20 mM Acetic acid buffer at pH 5.0 and 20 mM or 300 mM Trehalose. The concentration of the antibody is 120 mg/mL The results are shown in Table 10 above. In an embodiment, the formulation may include Acetic acid buffer, Trehalose, Methionine or NaCl, or a combination thereof, but not limited thereto.

TABLE 11

| | Trastu- | | | | | | | | |
|---|---|---|---|---|---|---|---|---|---|
| Sample | zumab | | | Mannitol | Met | Arg | NaCl | | |
| Test | Conc. | Temp. | | Conc. | Conc. | Conc. | Conc. | Size Exclusion-HPLC | |
| No. | (mg/ml) | (° C.) | Buffer | (mM) | (mM) | (mM) | (mM) | HMW (%) | |
| 36 | 120 | 40 | 20 mM | 20 | 20 | 0 | 0 | Initial | 0.8 |
| 37 | | | Ace | 20 | 20 | 50 | 0 | Initial | 2.15 |
| 38 | | | buffer | 300 | 20 | 0 | 0 | Initial | 0.74 |
| 39 | | | pH 5.0 | 20 | 20 | 0 | 150 | Initial | 0.94 |
| 40 | | | | 20 | 20 | 50 | 150 | Initial | 2.13 |
| 41 | | | | 20 | 0 | 0 | 0 | Initial | 0.85 |
| 42 | | | | 300 | 0 | 0 | 0 | Initial | 0.77 |

| Sample | | | | | | | | | | |
|---|---|---|---|---|---|---|---|---|---|---|
| Test | Size Exclusion-HPLC | | | | | | | | | |
| No. | HMW (%) | | Monomer (%) | | | | LMW (%) | | | |
| 36 | day 4 | 10.36 | Initial | 99.2 | day 4 | 86.48 | Initial | 0 | day 4 | 3.16 |
| 37 | day 4 | 100 | Initial | 97.85 | day 4 | 0 | Initial | 0 | day 4 | 0 |
| 38 | day 4 | 6.63 | Initial | 98.55 | day 4 | 90 | Initial | 0.71 | day 4 | 3.37 |
| 39 | day 4 | 38.09 | Initial | 99.06 | day 4 | 61.55 | Initial | 0 | day 4 | 0.36 |
| 40 | day 4 | 100 | Initial | 97.87 | day 4 | 0 | Initial | 0 | day 4 | 0 |
| 41 | day 4 | 11.7 | Initial | 99.15 | day 4 | 85.29 | Initial | 0 | day 4 | 3.01 |
| 42 | day 4 | 7.09 | Initial | 98.79 | day 4 | 89.98 | Initial | 0.44 | day 4 | 2.94 |

The above formulations were measured for stability after 0 days (Initial), and 4 days at a temperature of 40° C.±2° C. Formulations represented by samples 36 to 42 include 20 mM Acetic acid buffer at pH 5.0 and 20 mM or 300 mM Mannitol. The concentration of the antibody is 120 mg/mL The results are shown in Table 11 above. In an embodiment, the formulation may include Acetic acid buffer, Mannitol, Methionine or NaCl, or a combination thereof, but not limited thereto.

TABLE 12

| | Trastu- | | | | | | | | |
|---|---|---|---|---|---|---|---|---|---|
| Sample | zumab | | | Sorbitol | Met | Arg | NaCl | | |
| Test | Conc. | Temp. | | Conc. | Conc. | Conc. | Conc. | Size Exclusion-HPLC | |
| No. | (mg/ml) | (° C.) | Buffer | (mM) | (mM) | (mM) | (mM) | HMW (%) | |
| 43 | 120 | 40 | 20 mM | 20 | 20 | 0 | 0 | Initial | 0.68 |
| 44 | | | Ace | 20 | 20 | 50 | 0 | Initial | 1.92 |
| 45 | | | buffer | 300 | 20 | 0 | 0 | Initial | 0.47 |
| 46 | | | pH 5.0 | 20 | 20 | 0 | 150 | Initial | 0.55 |
| 47 | | | | 20 | 20 | 50 | 150 | Initial | 1.85 |
| 48 | | | | 20 | 0 | 0 | 0 | Initial | 0.77 |
| 49 | | | | 300 | 0 | 0 | 0 | Initial | 0.77 |

| Sample | | | | | | | | | | |
|---|---|---|---|---|---|---|---|---|---|---|
| Test | Size Exclusion-HPLC | | | | | | | | | |
| No. | HMW (%) | | Monomer (%) | | | | LMW (%) | | | |
| 43 | day 4 | 11.63 | Initial | 99.32 | day 4 | 85.3 | Initial | 0 | day 4 | 3.07 |
| 44 | day 4 | 100 | Initial | 98.08 | day 4 | 0 | Initial | 0 | day 4 | 0 |
| 45 | day 4 | 8.23 | Initial | 76.95 | day 4 | 91.07 | Initial | 22.58 | day 4 | 0.69 |
| 46 | day 4 | 40.89 | Initial | 86.34 | day 4 | 58.39 | Initial | 13.11 | day 4 | 0.72 |
| 47 | day 4 | 100 | Initial | 84.83 | day 4 | 0 | Initial | 13.31 | day 4 | 0 |
| 48 | day 4 | 13.24 | Initial | 73.73 | day 4 | 86.15 | Initial | 25.51 | day 4 | 0.6 |
| 49 | day 4 | 7.09 | Initial | 98.79 | day 4 | 89.98 | Initial | 0.44 | day 4 | 2.94 |

The above formulations were measured for stability after 0 days (Initial), and 4 days at a temperature of 40° C.±2° C. Formulations represented by samples 43 to 49 include 20 mM Acetic acid buffer at pH 5.0 and 20 mM or 300 mM Sorbitol. The concentration of the antibody is 120 mg/mL The results are shown in Table 12 above. In an embodiment, the formulation may include Acetic acid buffer, Sorbitol, Methionine or NaCl, or a combination thereof, but not limited thereto.

TABLE 13

| | Trastu- | | | | | | | | |
|---|---|---|---|---|---|---|---|---|---|
| Sample | zumab | | | Sucrose | Met | Arg | NaCl | | |
| Test | Conc. | Temp. | | Conc. | Conc. | Conc. | Conc. | Size Exclusion-HPLC | |
| No. | (mg/ml) | (° C.) | Buffer | (mM) | (mM) | (mM) | (mM) | HMW (%) | |
| 50 | 120 | 40 | 20 mM | 20 | 20 | 0 | 0 | Initial | 0.71 |
| 51 | | | Ace | 20 | 20 | 50 | 0 | Initial | 2.28 |
| 52 | | | buffer | 300 | 20 | 0 | 0 | Initial | 0.66 |
| 53 | | | pH 5.0 | 20 | 20 | 0 | 150 | Initial | 0.83 |
| 54 | | | | 20 | 20 | 50 | 150 | Initial | 2.18 |
| 55 | | | | 20 | 0 | 0 | 0 | Initial | 0.77 |
| 56 | | | | 300 | 0 | 0 | 0 | Initial | 0.76 |

| Sample | | | | | | | | | | |
|---|---|---|---|---|---|---|---|---|---|---|
| Test | Size Exclusion-HPLC | | | | | | | | | |
| No. | HMW (%) | | Monomer (%) | | | | LMW (%) | | | |
| 50 | day 4 | 10.8 | Initial | 98.94 | day 4 | 86.22 | Initial | 0.34 | day 4 | 2.98 |
| 51 | day 4 | 100 | Initial | 97.51 | day 4 | 0 | Initial | 0.21 | day 4 | 0 |
| 52 | day 4 | 4.71 | Initial | 99.22 | day 4 | 91.94 | Initial | 0.12 | day 4 | 3.34 |
| 53 | day 4 | 39.7 | Initial | 99.02 | day 4 | 58.03 | Initial | 0.15 | day 4 | 2.27 |
| 54 | day 4 | 100 | Initial | 97.69 | day 4 | 0 | Initial | 0.13 | day 4 | 0 |
| 55 | day 4 | 11.95 | Initial | 99.13 | day 4 | 85.4 | Initial | 0.1 | day 4 | 2.65 |
| 56 | day 4 | 7.35 | Initial | 99.15 | day 4 | 89.87 | Initial | 0.1 | day 4 | 2.79 |

It is worth noting that the formulation including sucrose has consistently high Monomer(%) after 0 days (Initial) over the formulation including sorbitol.

The above formulations were measured for stability after 0 days (Initial), and 4 days at a temperature of 40° C.±2° C. Formulations represented by samples 50 to 45 include 20 mM Acetic acid buffer at pH 5.0 and 20 mM or 300 mM Sucrose. The concentration of the antibody is 120 mg/mL The results are shown in Table 13 above. In an embodiment, the formulation may include Acetic acid buffer, Sucrose, Methionine or NaCl, or a combination thereof, but not limited thereto.

### Stability Studies Example 5: Composition of Stability Depending on Types of Amino Acids

Regarding the liquid pharmaceutical formulations used in Stabilities Studies Example 5, each buffer solution was prepared so as to be adapted for the corresponding amino acids, and antibody concentration, with addition of sucrose, and further addition of organic co-solvents, thus yielding the samples set forth in Tables 14 to 16 below. The concentration of each component is described in Tables 14 to 16 below. The total volume was, for example, 4 mL.

TABLE 14

| | Trastu- | | | | | Organic | | | |
|---|---|---|---|---|---|---|---|---|---|
| | zumab | | Sucrose | | AA | co- | | | |
| Example | Conc. | | Conc. | 10 mM | Conc. | solvent | Temp. | Storage | |
| No. | (mg/ml) | Buffer | (mM) | Met | (mM) | Conc. (%) | (° C.) | time | |
| 9 | 150 | 20 mM | 210 | + | — | 0.02% | 40 | Initial | |
| | | Ace | | | | PS80 | | 1 | wk |
| | | buffer | | | | | | 2 | wks |
| | | pH 5.2 | | | | | | 3 | wks |
| 10 | | | | | | | | 4 | wks |
| | | | | − | 50 mM | 0.02% | | Initial | |
| | | | | | Arg | PS80 | | 1 | wk |
| | | | | | | | | 2 | wks |
| | | | | | | | | 3 | wks |
| 11 | | | | | | | | 4 | wks |
| | | | | − | 20 mM | | | Initial | |
| | | | | | Glu | | | 1 | wk |
| | | | | | | | | 2 | wks |
| | | | | | | | | 3 | wks |
| | | | | | | | | 4 | wks |
| 12 | 150 | 20 mM | 210 | + | 50 mM | 0.02% | 40 | Initial | |
| | | Ace | | | Arg | PS80 | | 1 | wk |
| | | buffer | | | | | | 2 | wks |
| | | pH 5.2 | | | | | | 3 | wks |
| 13 | | | | | | | | 4 | wks |

| | | | |
|---|---|---|---|
| + | 20 mM | 0.02% | Initial |

| | | | |
|---|---|---|---|
| Glu | PS80 | 1 | wk |
| | | 2 | wks |
| | | 3 | wks |
| | | 4 | wks |

| | Size Exclusion-HPLC | | | Ion Exchange-HPLC | | |
|---|---|---|---|---|---|---|
| Example | HMW | Monomer | LMW | Acidic | Main | Basic |
| No. | (%) | (%) | (%) | variant(%) | Peak (%) | variant(%) |
| 9 | 0.86 | 99.14 | — | 28.04 | 63.78 | 8.18 |
| | 2.11 | 97.61 | 0.28 | 30.13 | 43.31 | 26.57 |
| | 2.49 | 97.19 | 0.33 | 33.61 | 35.42 | 30.97 |
| | 2.78 | 96.68 | 0.54 | 37.83 | 29.95 | 32.22 |
| 10 | 2.94 | 96.56 | 0.51 | 39.57 | 26.69 | 33.75 |
| | 1.03 | 98.97 | — | 28.15 | 64.16 | 7.69 |
| | 2.79 | 96.94 | 0.26 | 33.00 | 42.74 | 24.26 |
| | 3.5 | 96.23 | 0.28 | 38.87 | 33.62 | 27.51 |
| | 3.60 | 95.89 | 0.51 | 44.33 | 27.38 | 28.29 |
| 11 | 3.84 | 95.68 | 0.49 | 47.76 | 23.24 | 29.00 |
| | 0.90 | 99.10 | — | 28.47 | 63.91 | 7.62 |
| | 2.44 | 97.32 | 0.24 | 28.52 | 44.39 | 27.10 |
| | 2.97 | 96.69 | 0.34 | 31.43 | 36.17 | 32.40 |
| | 3.20 | 96.21 | 0.59 | 37.29 | 29.95 | 32.76 |
| | 3.44 | 96.04 | 0.53 | 39.57 | 25.78 | 34.65 |
| 12 | 0.98 | 99.02 | — | 28.69 | 64.33 | 6.98 |
| | 2.46 | 97.33 | 0.21 | 32.27 | 43.62 | 24.12 |
| | 2.99 | 96.70 | 0.31 | 37.70 | 35.62 | 26.67 |
| | 3.21 | 96.22 | 0.57 | 44.95 | 27.14 | 27.91 |
| 13 | 3.52 | 95.98 | 0.49 | 50.07 | 21.46 | 28.48 |
| | 0.92 | 99.08 | — | 28.32 | 63.93 | 7.75 |
| | 2.20 | 97.57 | 0.23 | 30.43 | 44.53 | 25.05 |
| | 2.57 | 97.13 | 0.31 | 31.80 | 36.45 | 31.75 |
| | 2.87 | 96.47 | 0.65 | 37.80 | 29.89 | 32.31 |
| | 3.19 | 96.27 | 0.54 | 41.16 | 26.02 | 32.82 |

The liquid pharmaceutical formulations prepared in Examples 9 to 13 were measured for stability after 0 weeks (marked as Initial), 1 week, 2 weeks, 3 weeks, and 4 weeks at a temperature of 40° C.±2° C. Examples 9 to 13 include 150 mg/mL trastuzumab, 210 mM Sucrose (or 7.18% w/v), and 0.02% PS80 in 20 mM Acetic acid buffer at pH 5.2. Example 9 further includes 10 mM Methionine, Example 10 further includes 50 mM Arginine. Example 11 further includes 20 mM Glutamic acid. Example 12 further includes a combination of 10 mM Methionine and 50 mM Arginine. Example 13 further includes a combination of 10 mM Methionine and 20 mM Glutamic acid. The results are shown in Table 14 above. In an embodiment, the formulation may include Acetic acid buffer, Sucrose, PS80, Methionine or Glutamic acid, or a combination thereof, but not limited thereto.

TABLE 15

| | | | Size Exclusion-HPLC | | | Ion Exchange-HPLC | | |
|---|---|---|---|---|---|---|---|---|
| Example | | Storage | HMW | Monomer | LMW | Acidic | Main | Basic |
| No. | Treatment | time | (%) | (%) | (%) | variant(%) | Peak (%) | variant(%) |
| 9 | Agitation | Initial | 0.86 | 99.14 | — | 28.04 | 63.78 | 8.18 |
| 13 | | 4 hrs | 1.17 | 98.83 | — | 29.09 | 61.82 | 9.09 |
| | | Initial | 0.92 | 99.08 | — | 28.32 | 63.93 | 7.75 |
| | | 4 hrs | 1.36 | 98.64 | — | 28.73 | 61.80 | 9.47 |

The liquid pharmaceutical formulations prepared in Examples 9 and 13 were measured for stability after 0 hours (marked as Initial) and after 4 hours under constant vortex for agitation stress test. The results are shown in Table 15 above. The formulations are stable after agitation.

TABLE 16

| | | | Size Exclusion-HPLC | | | Ion Exchange-HPLC | | |
|---|---|---|---|---|---|---|---|---|
| Example | | Storage | HMW | Monomer | LMW | Acidic | Main | Basic |
| No. | Treatment | time | (%) | (%) | (%) | variant(%) | Peak (%) | variant(%) |
| 9 | Freeze | Initial | 0.86 | 99.14 | — | 28.04 | 63.78 | 8.18 |
| | Thaw | 1 cycles | 0.84 | 99.16 | — | 28.69 | 63.59 | 7.72 |
| | | 3 cycles | 0.78 | 99.22 | — | 28.96 | 62.82 | 8.21 |
| | | 5 cycles | 0.72 | 99.29 | — | 29.24 | 63.94 | 6.82 |
| 13 | | Initial | 0.92 | 99.08 | — | 28.32 | 63.93 | 7.75 |
| | | 1 cycles | 0.92 | 99.08 | — | 28.97 | 63.09 | 7.95 |
| | | 3 cycles | 0.82 | 99.18 | — | 29.13 | 62.43 | 8.45 |
| | | 5 cycles | 0.78 | 99.22 | — | 29.15 | 64.20 | 6.65 |

The liquid pharmaceutical formulations prepared in Examples 9 and 13 were measured for stability after freeze-thaw 0 times (marked as Initial) 1 time, 3 times and 5 times (marked as cycles). The results are shown in Table 16 above. The formulations are stable after repeated freezing and thawing.

### Stability Studies Example 6: Composition of Stability Depending on Thermal or UV Stress

Regarding the liquid pharmaceutical formulations used in Stability Studies Example 6, each buffer solution was prepared so as to be adapted for the corresponding buffer concentration, pH and tonicity agents, with addition of Methionine, and further addition of organic co-solvents, thus yielding the samples set forth in Tables 17 to 18 below. The concentration of each component is described in Tables 17 to 18 below. The total volume was, for example, 5 mL.

TABLE 17

| | | | Trastu- | | | Organic | | | |
|---|---|---|---|---|---|---|---|---|---|
| | | | zumab | Sugar/ | Met | co- | | | |
| Example | | | Conc. | Polyol | Conc. | solvent | | Storage | |
| No. | Buffer | pH | (mg/ml) | (mM) | (mM) | (w/v %) | Treatment | time | |
| 14 | 20 mM | 6.0 | 120 | 210 mM | 10 | 0.04% | Thermal | Initial | |
| | L-Histidine/ | | | trehalose | | PS20 | (50 ± | 3 | days |
| | L-Histidine | | | | | | 2° C., | 6 | days |
| | hydro- | | | | | | 75 ± 5% | 10 | days |
| | chloride | | | | | | RH) | 2 | wks |
| 15 | | | | 200 mM | 8 | | | Initial | |
| | | | | sucrose | | | | 3 | days |
| | | | | | | | | 6 | days |
| | | | | | | | | 10 | days |
| | | | | | | | | 2 | wks |
| 16 | 20 mM | 5.0 | | 210 mM | 20 | | | Initial | |
| | Acetic | | | trehalose | | | | 3 | days |
| | Acid/ | | | | | | | 6 | days |
| | Sodium | | | | | | | 10 | days |
| | acetate | | | | | | | 2 | wks |

| | | | |
|---|---|---|---|
| 17 | 200 mM | 20 | Initial |

| | | |
|---|---|---|
| sucrose | 3 | days |
| | 6 | days |
| | 10 | days |
| | 2 | wks |

| | | | | | | | | FcγRIII |
|---|---|---|---|---|---|---|---|---|
| | | | | | | | | binding |
| | Size Exclusion-HPLC | | | | | DLS | | Relative |
| Example | HHMW | HMW | Monomer | LMW | LLMW | Radius | T onset | potency |
| No. | (%) | (%) | (%) | (%) | (%) | (nm) | (° C.) | (%) |
| 14 | 0 | 0.23 | 99.52 | 0.25 | 0 | 5.42 | 66.48 | 100 |
| | 0.01 | 0.58 | 98.77 | 0.63 | 0 | — | — | — |
| | 0.01 | 0.81 | 98.32 | 0.86 | 0 | — | — | 128.22 |
| | 0.02 | 1.06 | 97.82 | 1.11 | 0 | — | — | 54.34 |
| | 0.02 | 1.27 | 97.44 | 1.27 | 0 | — | — | — |
| 15 | 0.02 | 1.97 | 98.02 | 0 | 0 | 4.27 | 66.16 | 112.54 |
| | 0 | 2.48 | 97.52 | 0 | 0 | — | — | — |
| | 0 | 3.01 | 95.33 | 1.65 | 0 | — | — | 101.82 |
| | 0 | 3.57 | 94.4 | 2.03 | 0 | — | — | 108.95 |
| | 0 | 4.12 | 93.44 | 2.44 | 0 | — | — | — |
| 16 | 0.01 | 1.45 | 98.54 | 0 | 0 | 4.41 | 66.92 | 91.87 |
| | 0 | 2.21 | 97.79 | 0 | 0 | — | — | — |
| | 0 | 2.85 | 94.08 | 1 | 2.08 | — | — | 74.55 |
| | 0 | 3.49 | 92.8 | 1.34 | 2.36 | — | — | 66.67 |
| | 0 | 4.23 | 91.54 | 1.6 | 2.63 | — | — | — |
| 17 | 0.01 | 1.45 | 98.54 | 0 | 0 | 8.80 | 62.81 | 133.73 |
| | 0 | 2.21 | 97.79 | 0 | 0 | — | — | — |
| | 0 | 2.92 | 94.06 | 1 | 2.02 | — | — | 112.50 |
| | 0 | 3.97 | 91.9 | 1.56 | 2.57 | — | — | 134.82 |
| | 0 | 4.47 | 91.32 | 1.6 | 2.63 | — | — | — |

### Thermal Stress

The liquid pharmaceutical formulations prepared in Examples 14 to 17 were measured for stability after 0 days (marked as Initial), 3 days, 6 days, 10 days, and 2 weeks at a temperature of 50° C.±2° C. and 75±5% Room Humidity. The results are shown in Table 17 above. In an embodiment, the formulation may include Acetic acid buffer, Sucrose or Trehalose, Methionine, or a combination thereof, but not limited thereto. In another embodiment, the formulation may include Histidine buffer, Sucrose or Trehalose, Methionine, or a combination thereof, but not limited thereto.

TABLE 18

| | | | Trastu- | | | | | | |
|---|---|---|---|---|---|---|---|---|---|
| | | | zumab | Tonicity | Met | Organic | | | |
| Example | | | Conc. | agent | Conc. | co-solvent | | Storage | |
| No. | Buffer | pH | (mg/ml) | (mM) | (mM) | (w/v %) | Treatment | time | |
| 14 | 20 mM | 6.0 | 120 | 210 mM | 10 | 0.04% | UV/Vis | Initial | |
| | L-Histidine/ | | | trehalose | | PS20 | radiation | 3 | days |
| | L-Histidine | | | | | | (≥200 watt | 6 | days |
| | hydro- | | | | | | hours/m² in | 10 | days |
| | chloride | | | | | | the UV range | 2 | wks |
| 15 | | | | 200 mM | 8 | | (320-400 nm)) | Initial | |
| | | | | sucrose | | | | 3 | days |
| | | | | | | | | 6 | days |
| | | | | | | | | 10 | days |
| | | | | | | | | 2 | wks |
| 16 | 20 mM | 5.0 | | 210 mM | 20 | | | Initial | |
| | Acetic | | | trehalose | | | | 3 | days |
| | Acid/ | | | | | | | 6 | days |
| | Sodium | | | | | | | 10 | days |
| | acetate | | | | | | | 2 | wks |

| | | | |
|---|---|---|---|
| 17 | 200 mM | 20 | Initial |

| | | |
|---|---|---|
| sucrose | 3 | days |
| | 6 | days |
| | 10 | days |
| | 2 | wks |

| | | | | | | | | FcγRIII |
|---|---|---|---|---|---|---|---|---|
| | | | | | | | | binding |
| | Size Exclusion-HPLC | | | | | DLS | | Relative |
| Example | HHMW | HMW | Monomer | LMW | LLMW | Radius | T onset | potency |
| No. | (%) | (%) | (%) | (%) | (%) | (nm) | (° C.) | (%) |
| 14 | 0 | 0.22 | 99.78 | 0 | 0 | 5.42 | 66.48 | 132.30 |
| | 0 | 0.27 | 99.05 | 0 | 0.68 | — | — | — |
| | 2.48 | 11.09 | 86.27 | 0 | 0.15 | — | — | 82.32 |
| | 1.46 | 12.05 | 85.64 | 0 | 0.85 | — | — | 192.98 |
| | 7.52 | 21.03 | 69.94 | 0 | 1.52 | — | — | — |
| 15 | 0.02 | 1.97 | 98.02 | 0 | 0 | 4.27 | 66.16 | 81.88 |
| | 0.82 | 12.85 | 85.99 | 0.34 | 0 | — | — | — |
| | 4.67 | 21.63 | 71.32 | 1.94 | 0.42 | — | — | 91.79 |
| | 8.66 | 17.3 | 70.51 | 3.53 | 0 | — | — | 97.90 |
| | 30.6 | 22.82 | 43.35 | 2.99 | 0.23 | — | — | — |
| 16 | 0.01 | 1.45 | 98.54 | 0 | 0 | 4.41 | 66.92 | 90.57 |
| | 0.17 | 6.78 | 93.05 | 0 | 0 | — | — | — |
| | 0.86 | 9.22 | 87.51 | 2.42 | 0 | — | — | 59.09 |
| | 6.1 | 15.51 | 72.38 | 3.62 | 2.39 | — | — | 99.40 |
| | 11.45 | 18.44 | 65.71 | 3.72 | 0.68 | — | — | — |
| 17 | 0.01 | 1.45 | 98.54 | 0 | 0 | 8.80 | 62.81 | 75.38 |
| | 0.08 | 4.34 | 95.57 | 0 | 0 | — | — | — |
| | 0.33 | 6.02 | 93.65 | 0 | 0 | — | — | 137.41 |
| | 0.76 | 6.43 | 91.34 | 1.47 | 0 | — | — | 123.00 |
| | 4.46 | 14.12 | 81.42 | 0 | 0 | — | — | — |

### Photostability

The liquid pharmaceutical formulations prepared in Examples 14 to 17 were measured for stability after 0 days (marked as Initial), 3 days, 6 days, 10 days, and 2 weeks at a constant exposure to UV/Visible light radiation (≥200 watt hours/m²) in the UV range (320 nm-400 nm). The results are shown in Table 18 above. In an embodiment, the formulation may include Acetic acid buffer, Sucrose, Methionine, or a combination thereof, but not limited thereto.

### Stability Studies Example 7: Composition of Stability Depending on Types of Buffer and Tonicity Agents

Regarding the liquid pharmaceutical formulations used in Stability Studies Example 7, each buffer solution was prepared so as to be adapted for the corresponding buffer types and tonicity agents, with addition of Methionine, and further addition of organic co-solvents, thus yielding the samples set forth in Tables 19 to 20 below. The concentration of each component is described in Tables 19 to 20 below. The total volume was, for example, 5 mL or 4 mL in respect to Table 19 or Table 20.

TABLE 19

| | | | | | | | Organic | | |
|---|---|---|---|---|---|---|---|---|---|
| | | Trastu- | | | | | co- | | |
| | | zumab | | | | Stabilizer | solvent | | |
| Example | | Conc. | Temp. | Sugar/Polyol | | 10 mM | 0.04% | Storage | |
| No. | Buffer | (mg/ml) | (° C.) | Trehalose | Sucrose | Met | PS20 | time | |
| 18 | 20 mM | 120 | 40 | 210 mM | — | + | + | Initial | |
| | His pH | | | | | | | 1 | wk |
| | 6.0 | | | | | | | 2 | wks |
| | | | | | | | | 4 | wks |
| | | | | | | | | 8 | wks |
| | | | | | | | | 12 | wks |
| 19 | | | | — | 210 mM | + | + | Initial | |
| | | | | | | | | 1 | wk |
| | | | | | | | | 2 | wks |
| | | | | | | | | 4 | wks |
| | | | | | | | | 8 | wks |
| | | | | | | | | 12 | wks |
| 20 | 20 mM | | | 210 mM | − | + | + | Initial | |
| | Ace pH | | | | | | | 1 | wk |
| | 5.2 | | | | | | | 2 | wks |
| | | | | | | | | 4 | wks |
| | | | | | | | | 8 | wks |
| | | | | | | | | 12 | wks |

| | | | | | |
|---|---|---|---|---|---|
| 21 | — | 210 mM | + | + | Initial |

| | |
|---|---|
| 1 | wk |
| 2 | wks |
| 4 | wks |
| 8 | wks |
| 12 | wks |

| | | | | | | | ECD |
|---|---|---|---|---|---|---|---|
| | | | | | | | binding |
| | Size Exclusion-HPLC | | | Ion Exchange-HPLC | | | Relative |
| Example | HMW | Monomer | LMW | Acidic | Main | Basic | potency |
| No. | (%) | (%) | (%) | variant(%) | Peak (%) | variant(%) | (%) |
| 18 | 0.86 | 99.14 | — | 25.17 | 61.98 | 12.85 | 100.0 |
| | 1.81 | 98.03 | — | 32.34 | 45.25 | 22.41 | |
| | 2.41 | 97.21 | — | 43.92 | 27.79 | 28.29 | |
| | 2.39 | 96.23 | 1.38 | 65.64 | 14.68 | 19.68 | NA |
| | 3.15 | 94.18 | 2.64 | 75.56 | 5.58 | 18.86 | |
| | 3.96 | 92.25 | 3.73 | ND | ND | ND | |
| 19 | 0.84 | 99.16 | — | 25.65 | 61.93 | 12.42 | 111.1 |
| | 1.83 | 98.01 | — | 32.16 | 45.57 | 22.28 | |
| | 2.51 | 97.10 | — | 44.46 | 27.41 | 28.13 | |
| | 2.51 | 96.83 | 0.66 | 66.68 | 14.17 | 19.15 | NA |
| | 3.48 | 94.74 | 1.77 | 76.77 | 5.11 | 18.12 | |
| | 4.16 | 92.43 | 3.41 | ND | ND | ND | |
| 20 | 0.87 | 99.13 | — | 24.98 | 61.06 | 13.96 | 111.1 |
| | 1.82 | 98.03 | — | 25.14 | 49.36 | 25.50 | |
| | 2.41 | 97.21 | — | 29.26 | 33.53 | 37.20 | |
| | 2.47 | 96.87 | 0.66 | 51.60 | 21.58 | 26.82 | NA |
| | 3.06 | 93.11 | 6.43 | 57.62 | 9.34 | 33.05 | |
| | 3.82 | 92.58 | 3.60 | ND | ND | ND | |
| 21 | 0.90 | 99.10 | — | 24.70 | 61.52 | 13.78 | 104.2 |
| | 1.70 | 98.13 | — | 24.92 | 48.84 | 26.23 | |
| | 2.33 | 97.29 | — | 29.67 | 32.97 | 37.36 | |
| | 2.30 | 95.96 | 1.74 | 51.25 | 21.46 | 27.29 | NA |
| | 3.24 | 93.04 | 7.79 | 58.21 | 9.68 | 32.11 | |
| | 4.03 | 92.14 | 3.83 | ND | ND | ND | |

TABLE 20

| | | | | | | | Organic | | | | | | | | ECD |
|---|---|---|---|---|---|---|---|---|---|---|---|---|---|---|---|
| Ex- | | | | | | | co- | | | | | Ion Exchange-HPLC | | | binding |
| am- | | Conc. | | | | Stabilizer | solvent | | Size Exclusion-HPLC | | | Acidic | Main | Basic | Relative |
| ple | | (mg/ | Temp. | Tonicity agent | | 10 mM | 0.04% | Storage | HMW | Monomer | LMW | variant | Peak | variant | potency |
| No. | Buffer | ml) | (° C.) | Trehalose | Sucrose | Met | PS20 | time | (%) | (%) | (%) | (%) | (%) | (%) | (%) |
| 22 | 20 mM | 150 | 40 | 210 mM | − | + | + | Initial | 0.96 | 99.04 | − | 25.31 | 61.96 | 12.74 | 100.0 |
| | His | | | | | | | 1 wk | 2.02 | 97.82 | − | 32.95 | 43.42 | 23.64 | NA |
| | pH | | | | | | | 2 wks | 2.85 | 96.83 | − | 44.70 | 26.74 | 28.55 | |
| | 6.0 | | | | | | | 4 wks | 3.22 | 95.13 | 1.41 | 66.84 | 14.15 | 19.01 | |
| | | | | | | | | 8 wks | 4.00 | 93.34 | 2.87 | ND | ND | ND | |
| | | | | | | | | 12 wks | 4.89 | 91.37 | 3.66 | ND | ND | ND | |
| 23 | | | | − | 210 | + | + | Initial | 0.98 | 99.02 | − | 25.38 | 63.34 | 11.29 | 111.1 |
| | | | | | mM | | | 1 wk | 2.19 | 97.69 | − | 33.34 | 44.45 | 22.21 | NA |
| | | | | | | | | 2 wks | 2.94 | 96.70 | − | 45.55 | 26.46 | 27.99 | |
| | | | | | | | | 4 wks | 3.19 | 95.16 | 2.80 | 67.57 | 13.87 | 18.56 | |
| | | | | | | | | 8 wks | 4.18 | 92.42 | 4.24 | ND | ND | ND | |
| | | | | | | | | 12 wks | 5.17 | 91.14 | 4.26 | ND | ND | ND | |
| 24 | 20 mM | | | 210 mM | − | + | + | Initial | 0.86 | 99.14 | − | 24.98 | 62.70 | 12.32 | 111.1 |
| | Ace | | | | | | | 1 wk | 2.05 | 97.81 | − | 25.95 | 49.08 | 24.96 | NA |
| | pH | | | | | | | 2 wks | 2.85 | 96.75 | − | 30.12 | 33.27 | 36.61 | |
| | 5.2 | | | | | | | 4 wks | 3.04 | 95.40 | 1.55 | 52.61 | 20.84 | 26.55 | |
| | | | | | | | | 8 wks | 3.56 | 92.24 | 6.27 | 59.79 | 5.34 | 30.87 | |
| | | | | | | | | 12 wks | 4.54 | 92.05 | 3.50 | ND | ND | ND | |
| 25 | | | | − | 210 | + | + | Initial | 0.91 | 99.09 | − | 24.75 | 63.43 | 11.82 | 104.2 |
| | | | | | mM | | | 1 wk | 1.96 | 97.88 | − | 25.53 | 48.85 | 25.62 | NA |
| | | | | | | | | 2 wks | 2.87 | 96.69 | − | 29.62 | 33.27 | 37.11 | |
| | | | | | | | | 4 wks | 3.07 | 94.88 | 2.03 | 52.37 | 21.11 | 26.52 | |
| | | | | | | | | 8 wks | 3.86 | 93.53 | 3.52 | 58.57 | 9.87 | 31.55 | |
| | | | | | | | | 12 wks | 4.79 | 91.79 | 4.33 | ND | ND | ND | |

The liquid pharmaceutical formulations prepared in Examples 18 to 25 were measured for stability after 0 weeks (marked as Initial), 1 week, 2 weeks, 4 weeks, 8 weeks and 12 weeks at a temperature of 40° C.±2° C. Examples 18 to 21 are formulations including antibody concentration of 120 mg/mL. Examples 22 to 25 are formulations including antibody concentration of 150 mg/mL. The results are shown in Tables 19 and 20 above. In an embodiment, the formulation may include Acetic acid buffer, Methionine, Sucrose or Trehalose, or a combination thereof, but not limited thereto. In another embodiment, the formulation may include Histidine buffer, Methionine, Sucrose or Trehalose, or a combination thereof, but not limited thereto.

### Stability Studies Example 8: Composition of Stability Depending on Types of Buffer Organic Co-Solvents and Amino Acids

Regarding the liquid pharmaceutical formulations used in Stability Studies Example 8, each buffer solution was prepared so as to be adapted for the corresponding buffer types, organic co-solvents, and amino acids, with addition of tonicity agents, thus yielding the samples set forth in Tables 21 to 22 below. The concentration of each component was as described in Tables 21 to 22 below. The total volume was, for example, 5 mL or 4 mL in respect to Table 21 or Table 22.

TABLE 21

| | | Trastu- | | | | | | Size |
|---|---|---|---|---|---|---|---|---|
| | | zumab | | | | Organic | Storage | Exclusion-HPLC |
| Example | | Conc. | Temp. | Tonicity | Stabi- | co- | time | HHMW |
| No. | Buffer | (mg/ml) | (° C.) | agent | lizer | solvent | (month) | (%) |
| 26 | 20 mM | 120 | 40 | 210 mM | 10 mM | 0.04% | Initial | — |
| | His pH | | | Trehalose | Met | PS20 | 0.5 | — |
| | 6.0 | | | | | | 1 | — |
| | | | | | | | 3 | 0.19 |
| | | | | | | | 6 | 0.32 |
| 27 | 20 mM | | | 210 mM | 10 mM | 0.04% | Initial | — |
| | Ace | | | Sucrose | Met | PS20 | 0.5 | — |
| | pH 5.2 | | | | | | 1 | — |
| | | | | | | | 3 | 0.19 |
| | | | | | | | 6 | 0.63 |
| 28 | | | | 210 mM | 10 mM | 0.02% | Initial | — |
| | | | | Sucrose | Met | PS 80 | 0.5 | — |
| | | | | | | | 1 | — |
| | | | | | | | 3 | — |
| 29 | | | | 210 mM | 10 mM | 0.02% | Initial | — |
| | | | | Sucrose | Met and | PS 80 | 0.5 | — |
| | | | | | 10 mM | | 1 | — |
| | | | | | Glu | | 3 | — |

| | Size Exclusion-HPLC | | | | Ion Exchange-HPLC | | |
|---|---|---|---|---|---|---|---|
| Example | HMW | Monomer | Tailing | LMW | Acidic | Main | Basic |
| No. | (%) | (%) | (%) | (%) | variant(%) | Peak (%) | variant(%) |
| 26 | 0.81 | 99.19 | — | — | 29.46 | 61.14 | 9.39 |
| | 3.28 | 96.72 | — | — | 42.45 | 30.03 | 27.52 |
| | 3.45 | 95.12 | — | — | 50.74 | 20.72 | 28.55 |
| | 4.97 | 83.15 | 8.36 | 3.33 | NA | NA | NA |
| | 5.93 | 72.68 | 15.19 | 5.89 | NA | NA | NA |
| 27 | — | 99.27 | — | — | 29.68 | 61.59 | 8.74 |
| | — | 97.00 | — | — | 30.66 | 34.37 | 34.97 |
| | — | 96.18 | — | 0.76 | 31.78 | 26.49 | 41.73 |
| | 4.59 | 88.33 | 4.96 | 1.93 | NA | NA | NA |
| | 7.54 | 80.29 | 7.87 | 3.67 | NA | NA | NA |
| 28 | — | 99.29 | — | — | 28.42 | 62.33 | 9.25 |
| | 1.46 | 98.24 | — | 0.31 | 33.16 | 35.45 | 31.40 |
| | 2.45 | 96.93 | 3.12 | 1.32 | 40.13 | 24.02 | 35.85 |
| | 5.15 | 88.73 | 4.19 | 1.93 | NA | NA | NA |
| 29 | — | 99.33 | — | — | 27.21 | 62.47 | 10.32 |
| | 1.86 | 97.83 | — | 0.31 | 26.27 | 40.72 | 33.02 |
| | 2.46 | 96.91 | — | 0.62 | 32.29 | 31.24 | 36.47 |
| | 4.70 | 89.58 | 3.83 | 1.89 | NA | NA | NA |

TABLE 22

| | | | | | | Organic | Storage | Size Exclusion-HPLC | | | Ion Exchange-HPLC | | |
|---|---|---|---|---|---|---|---|---|---|---|---|---|---|
| Example | | Conc. | Temp. | Tonicity | Stabi- | co- | time | HMW | Monomer | LMW | Acidic | Main | Basic |
| No. | Buffer | (mg/ml) | (° C.) | agent | lizer | solvent | (month) | (%) | (%) | (%) | variant(%) | Peak (%) | variant(%) |
| 30 | 20 mM | 150 | 40 | 210 mM | 10 mM | 0.04% | Initial | 0.65 | 99.35 | 0.00 | 29.67 | 63.15 | 7.18 |
| | His pH | | | Trehalose | Met | PS20 | 0.5 | 2.41 | 96.88 | 0.71 | 33.95 | 35.87 | 30.18 |
| | 5.5 | | | | | | 1 | 2.99 | 96.00 | 1.01 | 39.58 | 25.02 | 35.40 |
| | | | | | | | 3.36 | 4.35 | 87.81 | 7.84 | NA | NA | NA |
| | | | | | | | 6 | 5.50 | 82.34 | 12.16 | NA | NA | NA |
| 31 | 20 mM | | | 210 mM | 10 mM | 0.02% | Initial | 0.64 | 99.36 | 0.00 | 29.30 | 62.73 | 7.97 |
| | Ace pH | | | Sucrose | Met | PS80 | 0.5 | 2.63 | 96.89 | 0.48 | 31.69 | 35.27 | 33.04 |
| | 5.2 | | | | | | 1 | 3.20 | 96.02 | 0.78 | 35.95 | 26.12 | 37.93 |
| | | | | | | | 3.36 | 5.42 | 89.63 | 4.95 | NA | NA | NA |
| | | | | | | | 6 | 7.59 | 84.82 | 7.59 | NA | NA | NA |
| 32 | | | | 210 mM | 10 mM | 0.02% | Initial | 0.60 | 99.40 | 0.00 | 29.41 | 62.63 | 7.96 |
| | | | | Sucrose | Met and | PS 80 | 0.5 | 2.15 | 97.29 | 0.56 | 27.26 | 33.22 | 39.52 |
| | | | | | 10 mM | | 1 | 2.64 | 96.55 | 0.81 | 28.69 | 25.42 | 45.89 |
| | | | | | Glu | | 3.36 | 4.66 | 89.75 | 5.59 | NA | NA | NA |
| | | | | | | | 6 | 7.62 | 83.56 | 8.83 | NA | NA | NA |

The liquid pharmaceutical formulations prepared in Examples 26 to 32 were measured for stability after 0 months (marked as Initial), 0.5 months, 1 month, 3 months (or 3.36 months in Table 28), and 6 months at a temperature of 40° C.±2° C. Examples 26 to 29 are formulations including antibody concentration of 120 mg/mL. Examples 30 to 32 are formulations including antibody concentration of 150 mg/mL. The results are shown in Tables 21 and 22 above. In an embodiment, the formulation may include Acetic acid buffer, Sucrose, PS80, Methionine and Glutamic acid, but not limited thereto. In another embodiment, the formulation may include Acetic acid buffer, Sucrose, PS80, Methionine or Glutamic acid, or a combination thereof. In yet another embodiment, the formulation may include Histidine buffer, Trehalose, PS20, Methionine, or a combination thereof.

### Stability Studies Example 9: Long-Term Stability

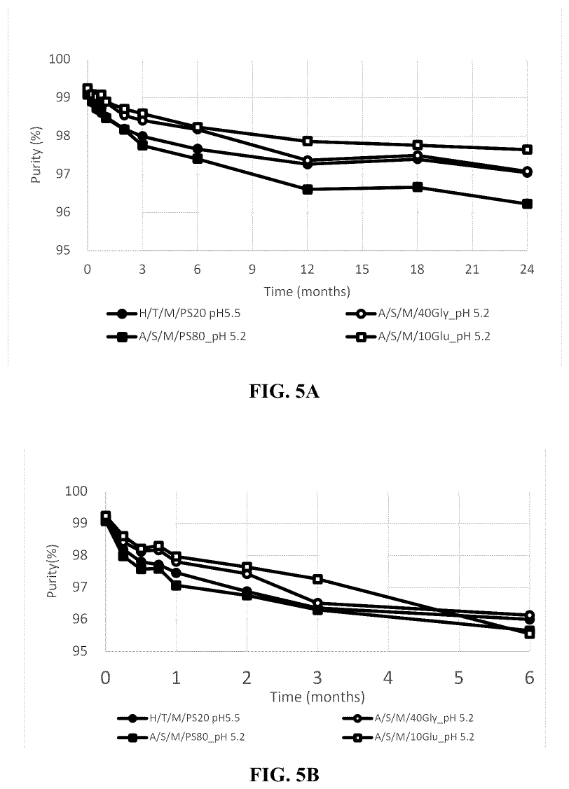

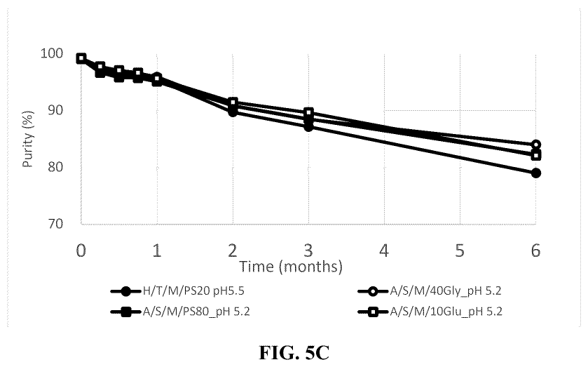

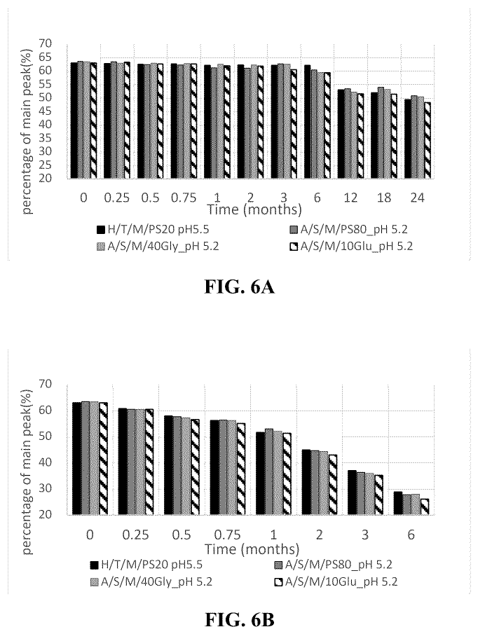

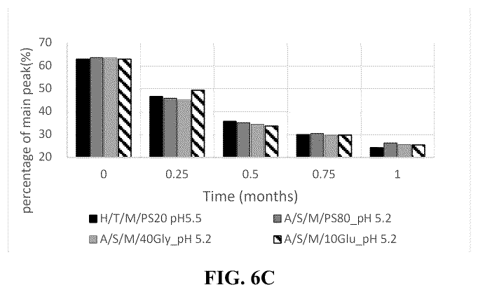

Regarding the liquid pharmaceutical formulations used in Stability Studies Example 9, each buffer solution was prepared so as to be adapted for the corresponding buffer types, organic co-solvents, and amino acids, with addition of tonicity agents, thus yielding the samples of Examples 33 to 36, which were measured for integrity stability by SEC-HPLC after 0, 0.25, 0.5, 0.75, 1, 2, 3.36, 6, 12, 18 and 24 months at 5° C., and up to 6 months at a temperature of 25° C. and 40° C. Example 33 is formulation including 150 mg/mL trastuzumab, 20 mM histidine buffer, pH 5.5, 210 mM trehalose, 10 mM methionine and 0.04% polysorbate 20 (H/T/M/PS20 pH5.5). Example 34 is formulation including 150 mg/mL trastuzumab, 20 mM acetic acid, pH 5.2, 210 mM sucrose, 10 mM methionine, and 0.02% polysorbate 80 (A/S/M/PS80 pH5.2). Example 35 is a formulation including 150 mg/mL trastuzumab, 20 mM acetic acid, pH 5.2, 210 mM sucrose, 10 mM methionine, 40 mM glycine and 0.02% polysorbate 80 (A/S/M/40Gly pH5.2). Example 36 is a formulation including 150 mg/mL trastuzumab, 20 mM acetic acid, pH 5.2, 210 mM sucrose, 10 mM methionine, 10 mM glutamic acid and 0.02% polysorbate 80 (A/S/M/10Glu pH5.2). The integrity results at the temperature of 5° C., 25° C. and 40° C. are shown in FIG. 5A, FIG. 5B and FIG. 5C respectively. The sample of Example 36 can maintain a high level of antibody purity for at least 24 months. The Examples 33 to 36 were measured for charge heterogeneity by CEX analysis at 5° C. after 0, 0.25, 0.5, 0.75, 1, 2, 3.36, 6, 12, 18 and 24 months (FIG. 6A), 6 months at the temperature of 25° C. (FIG. 6B) and 1 month at 40° C. (FIG. 6C).

### Stability Studies Example 10: Tonicity Agent Investigation

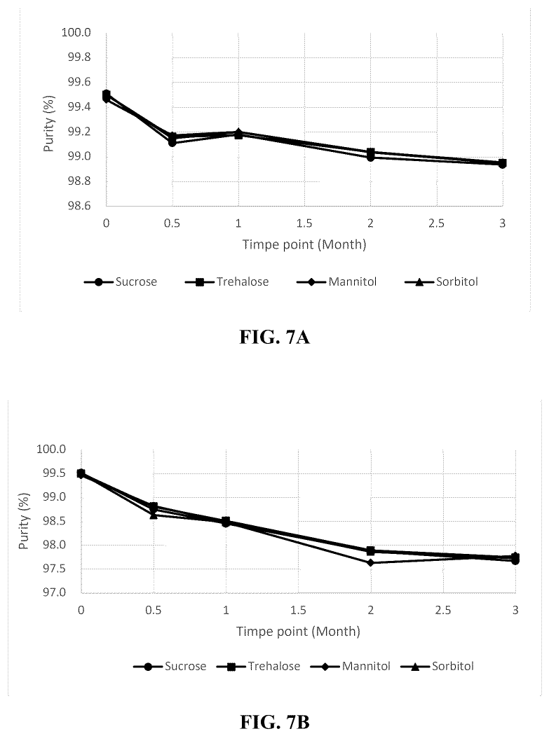

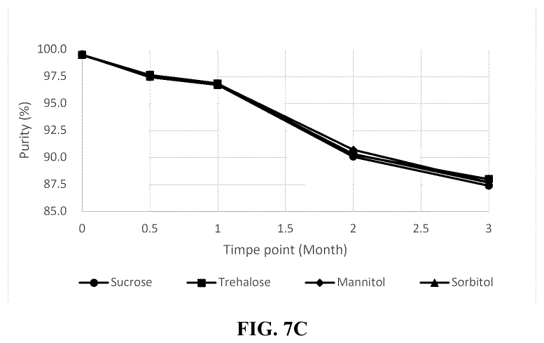

Regarding the liquid pharmaceutical formulations used in Stability Studies Example 10, each buffer solution was prepared so as to be adapted for the corresponding buffer types, organic co-solvents, and amino acids, with addition of tonicity agents, thus yielding the samples of Examples 37 to 40 were measured for integrity stability after 0, 0.5, 1, 2 and 3 months at a temperature of 5° C., 25° C. and 40° C. Example 37 is a formulation including 120 mg/mL trastuzumab, 20 mM acetic acid, pH 5.2, 210 mM sucrose, 10 mM methionine, 10 mM glutamic acid and 0.02% polysorbate 20. Example 38 is a formulation including 120 mg/mL trastuzumab, 20 mM acetic acid, pH 5.2, 210 mM trehalose, 10 mM methionine, 10 mM glutamic acid and 0.02% polysorbate 80. Example 39 is a formulation including 120 mg/mL trastuzumab, 20 mM acetic acid, pH 5.2, 210 mM mannitol, 10 mM methionine, 10 mM glutamic acid and 0.02% polysorbate 80. Example 40 is a formulation including 120 mg/mL trastuzumab, 20 mM acetic acid, pH 5.2, 210 mM sorbitol, 10 mM methionine, 10 mM glutamic acid and 0.02% polysorbate 80. The integrity results at the temperature of 5° C., 25° C. and 40° C. are shown in FIG. 7A, FIG. 7B, and FIG. 7C respectively.

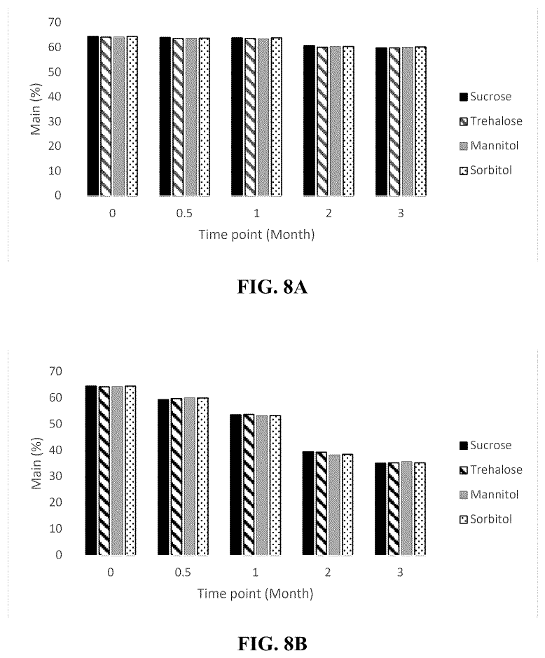

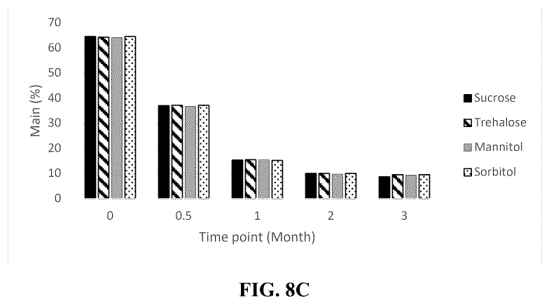

The liquid pharmaceutical formulations prepared in Examples 37 to 40 were measured for charge heterogeneity stability after 0, 0.5, 1, 2 and 3 months at a temperature of 5° C., 25° C. and 40° C. The charge heterogeneity results at the temperature of 5° C., 25° C. and 40° C. are shown in FIG. 8A, FIG. 8B, and FIG. 8C respectively.

### Stability Studies Example 11: Pertuzumab Stability Evaluation

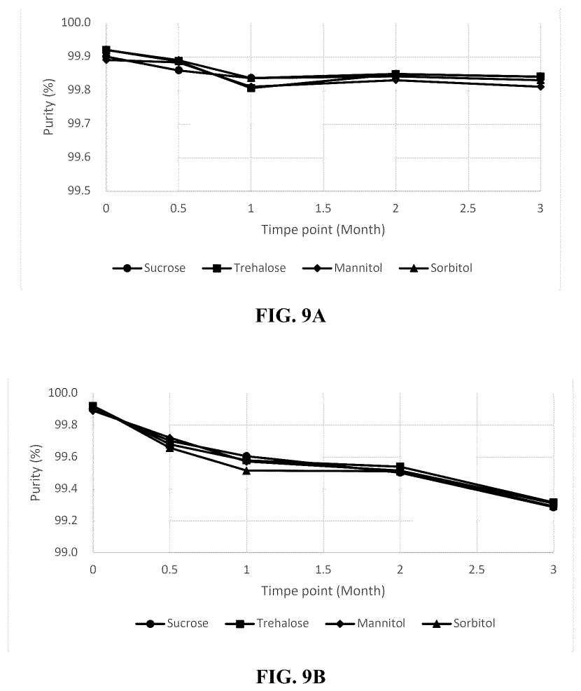

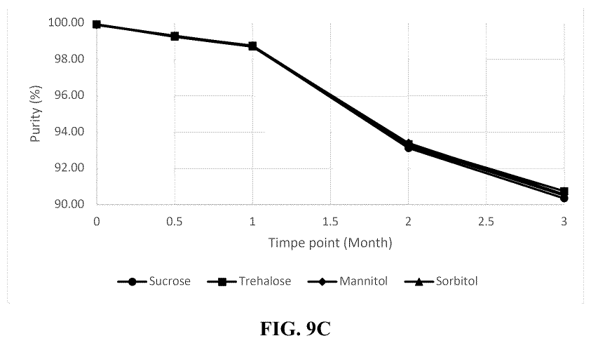

Regarding the liquid pharmaceutical formulations used in Stability Studies Example 11, each buffer solution was prepared so as to be adapted for the corresponding buffer types, organic co-solvents, and amino acids, with the addition of tonicity agents, thus yielding the samples of Examples 41 to 44 were measured for integrity stability after 0, 0.5, 1, 2 and 3 months at a temperature of 5° C., 25° C. and 40° C. Example 41 is a formulation including 120 mg/mL pertuzumab, 20 mM acetic acid, pH 5.2, 210 mM sucrose, 10 mM methionine, 10 mM glutamic acid and 0.02% polysorbate 20. Example 42 is a formulation including 120 mg/mL pertuzumab, 20 mM acetic acid, pH 5.2, 210 mM trehalose, 10 mM methionine, 10 mM glutamic acid and 0.02% polysorbate 80. Example 43 is a formulation including 120 mg/mL pertuzumab, 20 mM acetic acid, pH 5.2, 210 mM mannitol, 10 mM methionine, 10 mM glutamic acid and 0.02% polysorbate 80. Example 44 is a formulation including 120 mg/mL pertuzumab, 20 mM acetic acid, pH 5.2, 210 mM sorbitol, 10 mM methionine, 10 mM glutamic acid and 0.02% polysorbate 80. The integrity results at the temperature of 5° C., 25° C. and 40° C. are shown in FIG. 9A, FIG. 9B, and FIG. 9C respectively. The samples of Examples 41 to 44 can maintain a high level of antibody purity of pertuzumab.

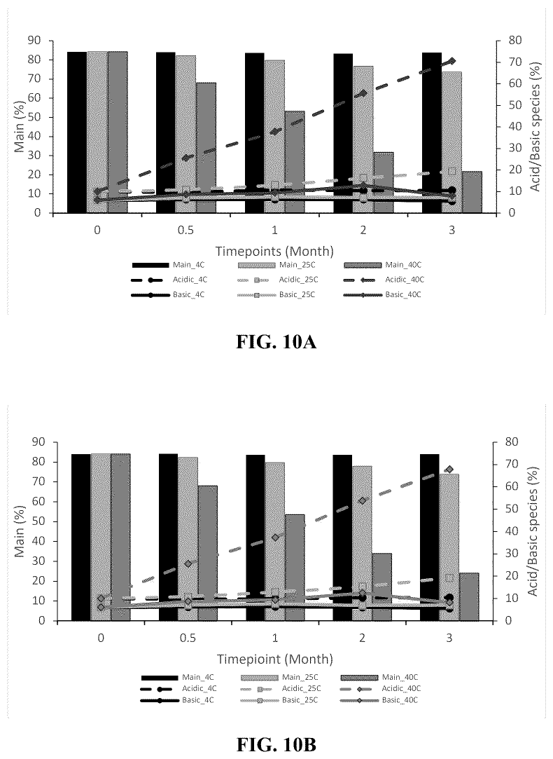

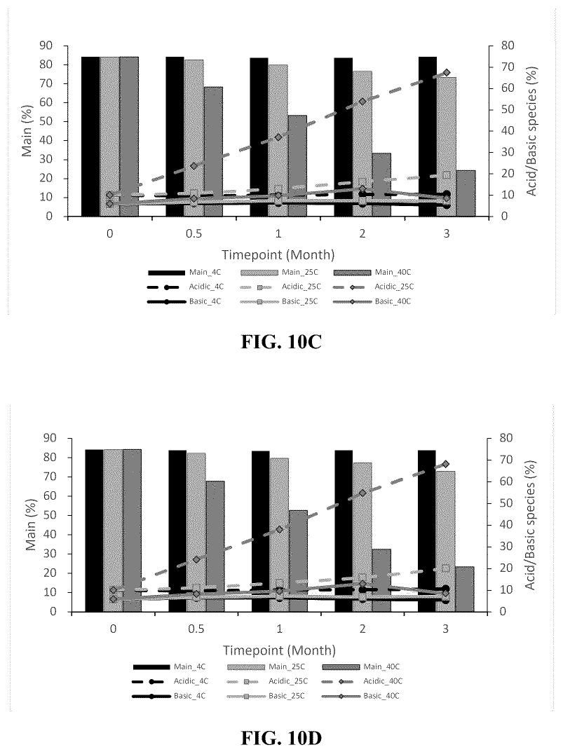

The liquid pharmaceutical formulations prepared in Examples 41 to 44 were measured charge heterogeneity stability after 0, 0.5, 1, 2 and 3 months at a temperature of 5° C., 25° C. and 40° C. The charge heterogeneity results of Examples 41 to 44 are shown in FIG. 10A, FIG. 10B, and FIG. 10C, and FIG. 10D respectively.

### Animal Studies

### Animal Studies Example 1: Mouse Pharmacodynamic (PD) Study—Antitumor Activity of Trastuzumab in Different Buffer Conditions.

### Xenograft Models.

All procedures were conducted in compliance with the applicable laws, regulations, and guidelines of the National Institutes of Health (NIH) and were approved by the Institutional Animal Care and Use Committee at Covance, U.S.A. BT-474 human breast carcinoma cells (ATCC, U.S.A.) (1.0E+007 BT-474) in 200 μL of serum-free Dulbecco's Modified Eagle Medium (DMEM) medium were inoculated S.C. into NSG mice (NOD.Cg-Prkdc^{side}Il2rg^{tm1Wjl}/SzJ) mice (Jackson Laboratory, U.S.A.). Treatment was commenced 7 days post tumor inoculation. Tumor volume in mm³ was determined using the formula (length×width²)/2, where length was the longest axis and width was the thickness measurement at right angles to the length. Data are expressed as mean tumor volume±SE for each treatment group. All data were analyzed for significance by a Student's T-test. Six mice per group were used in each study.

### Cell Lines and Culture Conditions

The human breast cancer cells BT-474 were cultured in DMEM, 10% Non-Heat-Inactivated Fetal Bovine Serum (FBS) and 1% Penicillin/Streptomycin/L-Glutamine (PSG) at 37° C. At implant in mice, the cell viability was 95%.

### Treatment of Animals

All mice were sorted into study groups based on the caliper estimation of tumor burden. Mice were treated according to Tables 23

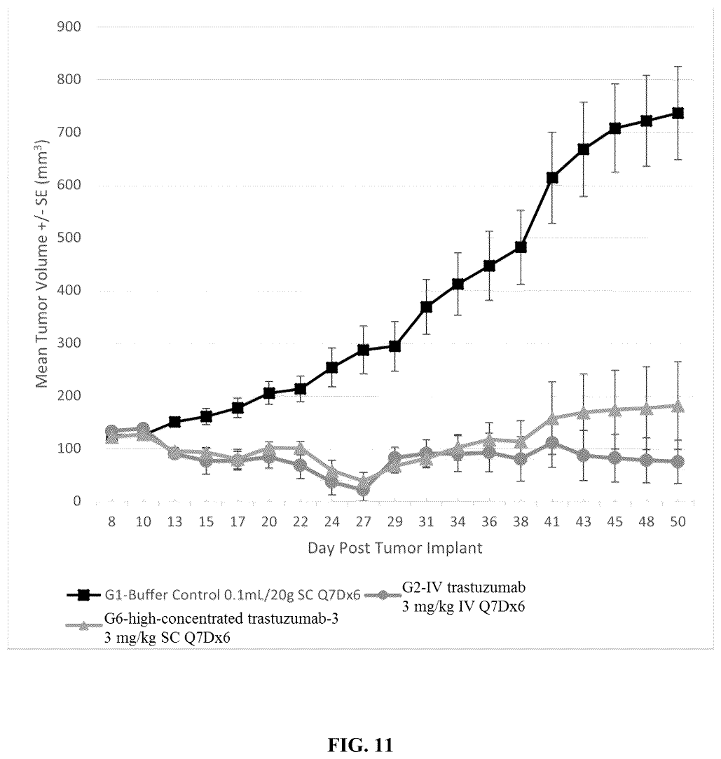

TABLE 23
Treatment groups and dosing schedules according to FIG. 11.

| | | Compound | | | | | |
|---|---|---|---|---|---|---|---|
| | | and | | | | | Days of |
| Group | N | Concentration | Formulation | Dose | ROA | Regimen | Treatment |
| 1 | 6 | None (Buffer | PBS | 0.1 mL/20 g | SC | Q7Dx6 | 8, 15, 22, |
| | | Control) | | | | | 29, 36, 43 |
| 2 | 6 | IV trastuzumab | IV formulation | 3 mg/kg | IV | Q7Dx6 | 8, 15, 22, |
| | | 21 mg/mL | | | | | 29, 36, 43 |
| 6 | 6 | 120 mg/mL | Formulation 3 | 3 mg/kg | SC | Q7Dx6 | 8, 15, 22, |
| | | high- | | | | | 29, 36, 43 |
| | | concentrated | | | | | |
| | | trastuzumab-3 | | | | | |

Note:
ROA—route of administration; PBS—Phosphate-Buffered Saline; Herceptin SC—Commercial Roche EU sourced trastuzumab 600 mg solution for SC injection, high-concentrated trastuzumab and IV trastuzumab are same active pharmaceutical ingredient (API) (trastuzumab); SC—subcutaneous; IV—intravenous; Q7Dx6—one treatment every 7 days for six total treatments. IV trastuzumab, a 150 mg lyophilized trastuzumab, was reconstituted by water for injection and under the buffer of 4.4 mM L-histidine/L-histidine HCl, pH 6.0, 1.71% trehalose dihydrate, 0.01% PS20 ultimately. high-concentrated trastuzumab-3 formulated in 20 mM Acetic acid/Sodium acetate, pH 5.2, 210 mM Sucrose, 10 mM Met, 10 mM Glu and 0.02% PS80

The PD results of these treatments can be found in FIG. 11.

Animal Studies Example 2: Mouse Pharmacodynamic (PD) Study to test plasma concentrations of trastuzumab (g/mL) vs. time in various formulations.

Test articles containing trastuzumab in various formulations for the PD study were prepared according to Table 24.

TABLE 24
Conditions in Mouse Pharmacokinetic (PK) Study

| | | | | | | | Organic co-solvent | |
|---|---|---|---|---|---|---|---|---|
| | | | Conc. | Polyol | | | 0.01% | 0.02% |
| Group | Formulation | Buffer | (mg/mL) | (w/v) | Stabilizer | | PS20 | PS80 |
| 3 | high- | 20 mM Acetic | 150 | 7.18% | 10 mM | 10 mM | − | + |
| | concentrated | acid/Sodium | | Sucrose | Met | Glu | | |
| | trastuzumab-3 | acetate pH 5.2 | | | | | | |
| −5 | IV | 20 mM | 150 | 1.71% | + | − | + | − |
| | trastuzumab | L-histidine/ | | Trehalose | | | | |
| | | L-histidine HCl | | dihydrate | | | | |
| | | pH 6.0 | | | | | | |

Note:
High-concentrated trastuzumab-3 and IV trastuzumab contain the same API (trastuzumab); Met—methionine; Ace—Acetic acid/Sodium acetate; PS—polysorbate.

The PK study was conducted in CD-1 male mice (Vital River, Pinghu, China). At the time of the study, mice were 5-6 weeks old and ranged in weight from 25-38 g. Animals were housed in groups of up to five animals/sex/cage in polycarbonate cages with bedding Animals were randomly allocated to each group. Mice were divided into five groups of 25 mice. Groups 1-4 were given SC bolus dose. Group 5 was given IV bolus dose. Animals were treated according to the protocol in Table 25.

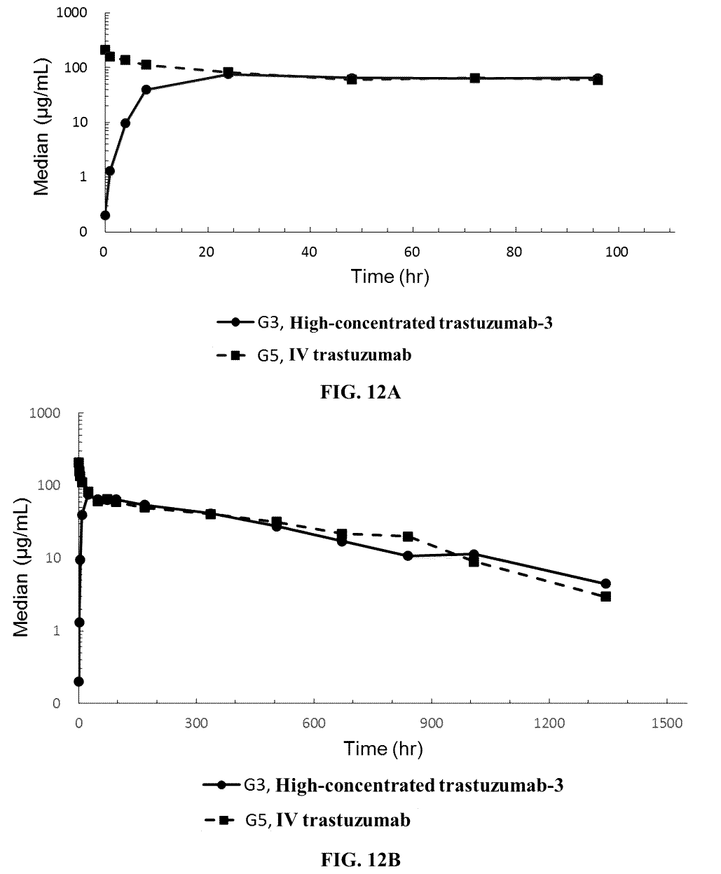

TABLE 25
Treatment groups according to FIG. 12.

| | | | No. of Animals | | Dose | Dose | Dose |
|---|---|---|---|---|---|---|---|
| | | Dose | and Sex | Dose | Level | Volume | Concentration |
| Group | Test Article | Route | (Male/Female) | Frequency | (mg/kg) | (mL/kg) | (mg/mL) |
| 3 | high- | SC | 25/0 | Single | 10 | 10 | 1 |
| | concentrated | | | | | | |
| | trastuzumab- | | | | | | |
| | 3 | | | | | | |
| 5 | IV | IV | 25/0 | Single | 10 | 5 | 2 |
| | trastuzumab | bolus | | | | | |

SC: Subcutaneous;
IV: Intravenous

About 0.2 mL blood per time point was collected from the submandibular sites. Samples were collected into serum separation tubes (SST) at 16-time points: Pre-dose and 0.25, 1, 4, 8, 24 hours post dose, and 48 (Day 2), 72 (Day 3), 96 (Day 4), 168 (Day 7), 336 (Day 14), 504 (Day 21), 672 (Day 28), 840 (Day 35), 1008 (Day 42) and 1344 (Day 56) hours post dose. No anticoagulant was used. Blood was allowed to clot for 30 minutes at room temperature. Samples were centrifuged at 2 to 8° C. for approximately 10 minutes at approximately 2700 g within 2 hours of collection, and serum was harvested and stored at −60 to −80° C. until further analysis.

### Bioanalysis.

Pharmacokinetics samples were analyzed for trastuzumab in mouse serum using ELISA method.

Results of the PK study are summarized in Table 26.

TABLE 26
Summary of the results of the PK study according to FIG. 12.

| | | | Dose | | | | | | | | | | |
|---|---|---|---|---|---|---|---|---|---|---|---|---|---|
| | Dose | | Level | T_{1/2} | Tmax | C₀ | Cmax | AUC₀₋ₗₐₛₜ | AUC_{0-inf} | V_{D} | CL | MRT | |
| Group | Route | Formulation | (mg/kg) | (hr) | (hr) | (μg/mL) | (μg/mL) | (hr*μg/mL) | (hr*μg/mL) | (mL/kg) | (mL/hr/kg) | (hr) | F % |
| 3 | SC | high- | 10 | 332 | 24.0 | NA | 75.7 | 34650 | 36798 | NA | NA | 478 | 92.3 |
| | | concentrated | | | | | | | | | | | |
| | | trastuzumab- | | | | | | | | | | | |
| | | 3 | | | | | | | | | | | |
| 5 | IV | IV | 10 | 186 | 0.250 | 229 | 209 | 37552 | 38354 | 70 | 0.261 | 419 | NA |
| | bolus | trastuzumab | | | | | | | | | | | |

Note:
SC: Subcutaneous; IV: Intravenous; T_{1/2}: Half-life of the sample to clear the animal body; C₀: Initial concentration; Tₘₐₓ: The amount of time for the drug is present at the maximum concentration in serum; Cₘₐₓ: The maximum (or peak) serum concentration of the drug; AUC₀₋ₗₐₛₜ: Area under the curve from time 0 to the last measurable concentration; AUC_{0-ifn}: Area under the curve to infinite time; V_{D}; Volume of distribution; CL: Clearance; MRT: Mean residence time; F %: Fraction bioavailability.
The results of the PK study are depicted in FIG. 12A and FIG. 12B.

### Equivalents and Scope

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise. This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A pharmaceutical composition comprising:
(a) an active pharmaceutical ingredient comprising anti-HER2 antibodies,
(b) methionine; and
(c) glutamic acid.

2. The pharmaceutical composition of claim 1, wherein the anti-HER2 antibodies comprising trastuzumab or pertuzumab.

3. The pharmaceutical composition of claim 1, wherein the anti-HER2 antibodies are trastuzumab, or pertuzumab and the trastuzumab or pertuzumab has a concentration from 80 to 150 mg/mL.

4. The pharmaceutical composition of claim 1, further comprising glycine, lysine, glutamine, or a combination thereof.

5. The pharmaceutical composition of claim 1, wherein the methionine has a concentration from 5 to 15 mM and the glutamic acid has a concentration from 5 to 15 mM.

6. The pharmaceutical composition of claim 1 further comprising a tonicity agent selected from trehalose, sucrose, mannitol, and sorbitol.

7. The pharmaceutical composition of claim 6, wherein the tonicity agent is sucrose.

8. The pharmaceutical composition of claim 7, wherein the sucrose has a concentration from 150 to 300 mM.

9. The pharmaceutical composition of claim 3, wherein the trastuzumab or pertuzumab has a concentration from 120 to 150 mg/mL.

10. The pharmaceutical composition of claim 1, wherein the composition is configured for subcutaneous administration.

11. The pharmaceutical composition of claim 1 further comprising acetate buffer at pH 4.6 to 6.5.

12. A method of treating cancer in a subject in need thereof comprising administering subcutaneously to the subject a therapeutically effective amount of the pharmaceutical composition of claim 1.

13. The method of claim 12, wherein the cancer is breast cancer.

14. The methods of claim 12, wherein the cancer is metastatic gastric or gastroesophageal junction adenocarcinoma.

15. The method of claim 12, wherein the pharmaceutical composition is administered once every three weeks.

16. A prefilled syringe comprising 3 mL to 7 mL of a solution, wherein the solution comprises:
(a) anti-HER2 antibody,
(b) methionine, and
(c) glutamic acid.

17. The prefilled syringe of claim 16, wherein the anti-HER2 antibody is trastuzumab or pertuzumab and has a concentration from 80 to 150 mg/mL.

18. The prefilled syringe of claim 17, wherein the methionine has a concentration from 5 to 15 mM and the glutamic acid has a concentration from 5 to 15 mM.

19. The prefilled syringe of claim 16, wherein the solution further comprising a sucrose.

20. A method of treating cancer in a subject in need thereof comprising administering subcutaneously to the subject a therapeutically effective amount of the solution from the prefilled syringe of claim 16.
